(19) United States
(12) Patent Application Publication
(10) Pub. No.: US 2025/0012555 A1
(43) Pub. Date: Jan. 9, 2025

(54) CURVE INDUCTIVE SENSOR

(71) Applicant: Magnisity Ltd., Hod-HaSharon (IL)

(72) Inventors: Ron BARAK, Tel-Aviv (IL); Benjamin GREENBURG, Hod-HaSharon (IL); Eyal KLEIN, Tel-Aviv (IL); Dror GARDOSH, Rishon LeTsiyon (IL)

(73) Assignee: Magnisity Ltd., Hod-HaSharon (IL)

(21) Appl. No.: 18/711,637

(22) PCT Filed: Nov. 21, 2022
(86) PCT No.: PCT/IL2022/051241

§ 371 Date: May 20, 2024

Related U.S. Application Data
(60) Provisional application No. 63/281,686, filed on Nov. 21, 2021.
(60) Provisional application No. 63/341,062, filed on May 12, 2022.
(60) Provisional application No. 63/406,787, filed on Sep. 15, 2022.

(51) Int. Cl. G01B 7/28; A61B 5/06; G01B 7/004; G01R 27/26; G01R 33/09

(52) U.S. Cl. CPC G01B 7/281; A61B 5/062; G01B 7/004; G01R 27/2611; G01R 33/096

(57) ABSTRACT

Flexible, longitudinally extended sensors comprising variably inductive sensing material. Sensors connect to an external processing unit using as few as 2 connections. In operation, sensors measure inductance along their longitudinal extent (curve). Spectral de-multiplexing allows electrical property differences along sensors to be resolved. This enables shape tracking of the flexible sensor. The sensor principles are suitable for the production of ultra-small diameter probes (for example, smaller than 0.5 mm) of arbitrary length (for example, 30 cm long). There are potential advantages for cost efficiency and ease of manufacture.

### RELATED APPLICATIONS

This application claims the benefit of priority under 35 U.S.C. § 119 (c) of U.S. Provisional Patent Application No. 63/281,686 filed Nov. 21, 2021; U.S. Provisional Patent Application No. 63/341,062 filed May 12, 2022; and of U.S. Provisional Patent Application No. 63/406,787 filed Sep. 15, 2022; the contents of which are incorporated herein by reference in their entirety.

### FIELD AND BACKGROUND OF THE INVENTION

The present invention, in some embodiments thereof, relates to the field of microprobe position and/or shape sensing and more particularly, but not exclusively, to sensing of probe positions and/or shapes using electrical field measurements.

Certain physical quantities can be measured by measuring the electrical resistance of a conductor affected by the physical phenomenon. For example, a resistance thermometer comprises a material which has an accurate resistance/temperature relationship which is used to provide indication of the temperature. In a strain gauge, the strain of an object is computed by measuring the electrical resistance of a foil attached to an object. As the object is deformed, the electrical resistance of the deformed foil changes which provides indication of the strain. Similarly, a force/pressure sensor uses a force-sensitive resistor to measure the force applied to the sensor. A magnetoresistive sensor comprises a foil (for example, permalloy, supermalloy, mu-metal, or cobalt alloy) which changes its resistance due to an externally applied magnetic field. The magnetoresistive sensor measures the resistance of the foil to compute the magnetic field at the position and orientation of the sensor in space by using a known resistance/magnetic field relationship.

A magneto-inductive sensor measures the inductance of a coil wrapped around a high permeability non-linear magnetic core (such as permalloy, supermalloy, mu-metal etc.) to compute the magnetic field at the position and orientation of the sensor in space by using a known inductance/magnetic field relationship.

To measure the electrical resistance of a conductor to a sufficient precision to detect and quantify variable resistive effects, a Wheatstone bridge is commonly used. The Wheatstone bridge converts the electrical resistance to be measured into a differential voltage quantity which can then be amplified, filtered and sampled with an ADC (Analog Digital Converter). A precisely known relation between the measured electrical resistance and the physical quantity (e.g., temperature, strain, force/pressure, magnetic field strength) is used in order to convert the measured resistance into a measurement of the desired physical quantity.

Several methods exist for measuring the inductance of an inductor to a precision sufficient to detect magneto-inductive changes. Some inductors have rather constant inductance L of some range of currents and frequencies, and can be measured for example using oscillation-based methods: for example, the inductor can be placed in a known RLC-type circuit and the resonance frequency f can be measured, which depends on the inductance of the inductor (as well as on values of R and C, which may be accounted for as known values). By knowing the frequency to inductance's (f-L) exact relationship, the inductance can be solved. A precisely known relation between the measured electrical resistance or inductance and the physical quantity (e.g., temperature, strain, force/pressure, or magnetic field) is used in order to convert the measured resistance or inductance into a measurement of the desired physical quantity.

In U.S. Pat. No. 9,658,298, a 3-axis magnetoresistive sensor is described which senses the magnetic field along 3 non-coplanar axes. The sensed quantities are then converted into a full 3-dimensional magnetic field measurement at the position and orientation of the sensor by applying calibration matrices, to convert the field measured in potentially non-orthogonal axes and non-unity gains to the sensor's orthonormal axes with unity gains.

In U.S. Pat. Publication No. 2013/0009635 A1 a magneto-inductive sensor is described which senses the magnetic field along magneto-inductive coils. The sensor measures the inductance of discrete coils, wrapped around a high permeability magnetic core, and converts the measured inductances to magnetic field measurements. The inductance is measured using digital oscillation techniques.

### SUMMARY OF THE INVENTION

According to an aspect of some embodiments of the present disclosure, there is provided a system configured to sense a sensed magnetic field, at a plurality of locations along a sensing region of a flexible and elongated probe body, the system including: the elongated probe body; a sensing circuit including serially interconnected inductor coil windings extending along the sensing region extending longitudinally along the elongated probe body; wherein a total variable electrical inductance of the sensing circuit integrates inductance density along the sensing region, the inductance density including: a baseline inductance density, distributed non-uniformly along the sensing region, sensing inductance density variations responsive to strength of the sensed magnetic field, and internal inductance density variations responsive to strength of an internal magnetization field generated by electrical current passing through the inductor coil windings; a readout controller configured to: provide electrical current to the sensing circuit at a plurality of electrical current values which respectively impose different internal inductance density variations, and receive electrical signals elicited in the inductor coil windings by the electrical current, and indicative of measurements of the variable electrical inductance; and a processor configured to: receive the measurements of the variable electrical inductance, receive the plurality of electrical current values respectively corresponding to the measurements, and calculate strengths of sensed magnetic field for the plurality of locations along the sensing region, using the electrical current values and corresponding measurements of the variable electrical inductance.

According to some embodiments of the present disclosure, the plurality of locations along the probe body comprise locations with respectively different inductance densities, such that the different electrical current values generate different respective internal magnetization field strengths in each location.

According to some embodiments of the present disclosure, the respectively different internal magnetization field strengths result in different internal inductance density variations in each of the plurality of locations along the sensing body.

According to some embodiments of the present disclosure, the different internal inductance density variations adjust the sensing inductance density variations to different functions of sensed magnetic field strength in each of the plurality of locations.

According to some embodiments of the present disclosure, the processor is configured to: receive a model of the non-uniform distribution of baseline inductance density; and calculate strengths of the sensed magnetic field to be consistent with both the internal magnetization field strengths, and the different functions of sensed magnetic field strength in each of the plurality of locations along the sensing body.

According to some embodiments of the present disclosure, the inductor coil windings wrap a magnetic inductor core which changes non-linearly in electromagnetic permeability as a function of total local magnetic field strength.

According to some embodiments of the present disclosure, material of the magnetic core has a high permeability of about 100000.

According to some embodiments of the present disclosure, the magnetic core includes at least one of the group consisting of: a permalloy, a supermalloy, a cobalt alloy, and mu-metal.

According to some embodiments of the present disclosure, the inductor coil windings along the sensing region comprise wire having a thickness between 40 AWG and 54 AWG.

According to some embodiments of the present disclosure, non-uniformity of the baseline inductance density is provided using a non-uniform density of coil windings along the sensing region.

According to some embodiments of the present disclosure, the non-uniform density of coil windings along the sensing region includes gradually changing coil spacing along the sensing region.

According to some embodiments of the present disclosure, the non-uniform density of coil windings along the sensing region includes discrete sections of coil along the sensing region, each section having a different number of coil windings.

According to some embodiments of the present disclosure, different numbers of coils windings are provided as coils in different numbers of layers.

According to some embodiments of the present disclosure, the plurality of electrical current values comprise at least 8 current values.

According to some embodiments of the present disclosure, the plurality of electrical current values comprise electrical current values between about −100 mA and about 100 mA.

According to some embodiments of the present disclosure, the plurality of electrical current values are provided as a repeating waveform signal.

According to some embodiments of the present disclosure, the repeating waveform signal includes one of the group of waveforms consisting of: sine wave, triangular wave, and sawtooth wave.

According to some embodiments of the present disclosure, the sensing region includes coil windings distributed along a sensing region length of between 40 mm and 200 mm.

According to some embodiments of the present disclosure, a maximum diameter of the elongated probe body along the sensing region is about 1.5 mm.

According to some embodiments of the present disclosure, a ratio between largest and smallest diameters along the sensing region is at least 1.5.

According to some embodiments of the present disclosure, non-uniformity of the baseline inductance density is provided using a range of different diameters between the largest and smallest diameter as diameters of different inductive coil windings contributing to the total inductance.

According to some embodiments of the present disclosure, non-uniformity of the baseline inductance density is provided using variations in composition of a magnetic core of the inductor coils contributing to the total inductance.

According to some embodiments of the present disclosure, the processor is configured to calculate strengths of sensed magnetic field for the plurality of locations along the sensing region by solving a system of non-linear equations, each respectively defined using the measurements of the variable electrical inductance and their corresponding electrical current values.

According to some embodiments of the present disclosure, the system includes a magnetic field generating controller and a plurality of magnetic field generating transducers generating a respective plurality of sensed magnetic fields including and each measured as claimed for the sensed magnetic field.

According to some embodiments of the present disclosure, the plurality of sensed magnetic fields are generated at different frequencies, and distinguished by the readout controller that provides the measurements using differences in their frequencies.

According to some embodiments of the present disclosure, the system includes a processing unit configured to determine a shape and position of the sensing circuit, using the measurements of sensed magnetic field strengths, and estimated positions of regions exposed to the magnetic field strengths from the magnetic field transducers.

According to some embodiments of the present disclosure, the sensing circuit is integrated into an endoluminal device having a length to diameter ratio of at least 100.

According to some embodiments of the present disclosure, components of the sensing circuit also provide mechanical properties of the endoluminal device at a distal end thereof, the mechanical properties being suitable for advancing the sensing circuit by insertion of the endoluminal device into a body cavity a distance of at least 50 mm, to reach a body cavity region having an inner diameter of 5 mm or less.

According to some embodiments of the present disclosure, the provided measurements are constrained by criteria requiring one or more of the group consisting of: smoothness of the sensed magnetic field along the sensing region, preservation of distances between sensing regions, and/or preservation of known positions of sensing regions.

According to some embodiments of the present disclosure, the inductor coil windings surround an inductor core material; and the processor is configured to: access at least one impedance curve defining changes in inductance density as a function of magnetization of the inductor core material, and calculate the strengths of sensed magnetic field for the plurality of locations along the sensing region, using the impedance curve.

According to an aspect of some embodiments of the present disclosure, there is provided a method of sensing a sensed magnetic field at a plurality of locations along a flexible elongated probe body, the method including: sensing total inductances of a flexible linear sensing circuit for each of a plurality of different electrical current values passed through the circuit; and determining the sensed magnetic field for a plurality of mutually distinguished locations along the flexible linear sensing circuit, using: the total inductances, the electrical current values respectively associated with each total inductance, a model of non-uniformities in the distribution of inductance density along the sensing circuit which distinguish the regions, and a model of non-linearities in the inductances in the regions in response to exposure to magnetic fields.

According to some embodiments of the present disclosure, the method includes using the sensed magnetic field to determine one or more positions along the flexible elongated probe body.

According to some embodiments of the present disclosure, the method includes using the sensed magnetic field to determine a shape of the flexible elongated probe body.

According to an aspect of some embodiments of the present disclosure, there is provided a system configured to sense one or more sensed magnetic fields, including: an endolumenal medical device, including a probe with a sensing region; the sensing region having a variable electrical inductance provided by an inductor coil surrounding an inductor core of a highly permeable and non-linear material; wherein a measurement of the variable inductance includes contributions from: a baseline inductance, sensing inductance variations responsive to strength of the one or more sensed magnetic fields, and internal inductance variations responsive to magnetization by current passing through the inductor coil; a readout controller configured to: pass current through the inductor coil to impose internal inductance variation, and receive electrical signals elicited in the inductor coil by the current, and indicative of measurements of the variable inductance; and a processor configured to: receive the measurements of the variable inductance, receive current values corresponding to current passed through the inductor coil to impose internal inductance variation, and calculate strength of the one or more sensed magnetic fields using the measurements of the variable inductance.

According to some embodiments of the present disclosure, the processor calculates strength of the one or more sensed magnetic fields also using the baseline inductance, and sensing inductance variations calculated from the current values.

According to some embodiments of the present disclosure, the processor is also configured to: access a model describing distributions of the one or more sensed magnetic fields through a region of space, and determine a position of the sensing region, using the calculated strengths of the one or more sensed magnetic fields and the model.

According to some embodiments of the present disclosure, the sensing region is elongated along a longitudinal axis of the probe; the inductor coil surrounding the inductor core is constructed to provide a variable electrical inductance including integrated inductance density having an inhomogeneous distribution along the longitudinal axis; the readout controller passes current at a plurality of values; and the processor is configured to: access a model of the inhomogeneous distribution of inductance density, and calculate strength of the one or more sensed magnetic fields for distinguishable regions of the sensing region along the longitudinal axis, using the model of the inhomogeneous distribution of inductance density.

According to an aspect of some embodiments of the present disclosure, there is provided a system configured to sense position in space and/or shape along a flexible elongated probe body, the system including: the elongated probe body; a sensing circuit including an electrical conductor extending between a first end and a second end through a sensing region extending longitudinally along the elongated probe body; a readout controller configured to pass an electrical current through the electrical conductor between the first and second ends, and obtain measurements of an electrical signal produced in response; wherein a plurality of locations along the sensing region are structured to electrically interact with the electrical current both: in a manner dependent on local magnetic field, and inhomogeneously compared to each other, regardless of local magnetic field; and a processor configured to: receive the measurements from the readout controller, receive one or more models describing how the plurality of locations are structured, receive a model of magnetic fields in a region surrounding the probe body, calculate local magnetic fields for the plurality of locations, using the measurements and the one or more models, and calculate positions of the plurality of locations, by comparing the calculated magnetic fields with the model of magnetic fields.

According to some embodiments of the present disclosure, the plurality of locations are structured inhomogeneously by differences in inductance along the sensing region.

According to some embodiments of the present disclosure, the plurality of locations are structured inhomogeneously by differences in resistance along the sensing region.

According to some embodiments of the present disclosure, the plurality of locations are structured inhomogeneously by differences in capacitance along the sensing region.

According to some embodiments of the present disclosure, the plurality of locations comprise a magnetoresistive element that interacts with the electrical current according to a resistance modified by local magnetic field.

According to some embodiments of the present disclosure, the plurality of locations comprise a magnetoinductive element that interacts with the electrical current according to a permeability modified by local magnetic field.

According to an aspect of some embodiments of the present disclosure, there is provided a system configured to sense an environmental parameter at a plurality of locations along a longitudinally extended probe body, the system including: the longitudinally extended probe body; a sensing circuit including a sensing region extending longitudinally along the probe body, and electrically interconnecting a pair of electrical terminals through a total resistance of the sensing region; wherein the total resistance of the sensing region integrates resistances through portions of material spaced along the sensing region and having variable resistivity in response to changes in an environmental parameter; and a readout controller, the readout controller being configured to: read electrical signals from the terminals, the electrical signals being encoded as amplitude and phase modulations at a plurality of frequencies, and provide measurements of the environmental parameter for a plurality of locations along the sensing region, based on resistivity information encoded in the electrical signals.

According to some embodiments of the present disclosure, the readout controller distinguishes resistivities among the portions of material different resistivity, and the measurements are determined based on the distinguished resistivities.

According to some embodiments of the present disclosure, the sensing circuit includes a reactive load distributed along the sensing region, and the amplitude modulations differ among the plurality of frequencies in part according to the distribution of the reactive load.

According to some embodiments of the present disclosure, the plurality of frequencies are determined by inputs to the sensing circuit from the readout controller.

According to some embodiments of the present disclosure, the inputs are provided as a multispectral signal.

According to some embodiments of the present disclosure, the multispectral signal is a square wave.

According to some embodiments of the present disclosure, the readout controller distinguishes the resistivities based on differential contributions of the portions of material to the amplitude modulations, according to frequency.

According to some embodiments of the present disclosure, the reactive load includes reactances distributed continuously along the sensing region.

According to some embodiments of the present disclosure, the reactances comprise intrinsic self-capacitance and self-inductance of conductors of the sensing region.

According to some embodiments of the present disclosure, the reactive load includes reactances distributed in discrete components along the sensing region.

According to some embodiments of the present disclosure, the discrete components sub-divide the sensing circuit to create resonant peaks at different frequencies, and the portions of material contribute differentially from each other in determining the resonant peak amplitudes.

According to some embodiments of the present disclosure, the readout controller localizes resistivities using the resistivity information and known values of the reactances.

According to some embodiments of the present disclosure, the readout controller converts the resistivities into the measurements of the environmental parameter, based on known effects of the environmental parameter on resistivity of the portions of material.

According to some embodiments of the present disclosure, the variable resistivity is variable in response to changes in magnetic field interacting with the portions of material.

According to some embodiments of the present disclosure, the variable resistivity is variable in response to changes in temperature of the portions of material.

According to some embodiments of the present disclosure, the variable resistivity is variable in response to changes to the strain of the portions of material.

According to some embodiments of the present disclosure, the measurements of the environmental parameter are local measurements of the magnetic fields generated by the magnetic field generating transducers.

According to some embodiments of the present disclosure, the magnetic fields are arranged to produce characteristic patterns of magnetic field amplitude and frequency which distinctively characterize spatial locations in a region containing the sensing circuit.

According to some embodiments of the present disclosure, the readout controller matches the measurements of the environmental parameter to characteristic patterns of magnetic field amplitude and frequency expected to be generated by the transducers.

According to some embodiments of the present disclosure, the readout controller determines a shape and position of the sensing circuit, using the matched measurements and characteristic patterns.

According to some embodiments of the present disclosure, the magnetic field generating transducers generate at least 30 alternating magnetic fields.

According to some embodiments of the present disclosure, the sensing circuit includes flexible printed circuit (FPC) embedding the portions of material, and the portions of material comprise a variably resistive film.

According to some embodiments of the present disclosure, the variably resistive film is divided into discrete film units connected in series.

According to some embodiments of the present disclosure, each discrete unit of variably resistive film is etched in an FPC manufacturing process.

According to some embodiments of the present disclosure, each discrete unit of variably resistive film includes a plurality of substantially parallel stripes, interconnected at alternating ends.

According to some embodiments of the present disclosure, each discrete unit of variably resistive film is embedded inside a separately packaged component.

According to some embodiments of the present disclosure, the FPC is interconnected with the readout controller through a number of wires less than twice the number of discrete units.

According to some embodiments of the present disclosure, the FPC is interconnected with the readout controller through a number of wires less than the number of discrete units.

According to some embodiments of the present disclosure, the FPC is interconnected with the readout controller through two wires.

According to some embodiments of the present disclosure, the sensor contains reactive components between the film units.

According to some embodiments of the present disclosure, the sensor does not contain any discrete reactive components.

According to some embodiments of the present disclosure, the discrete film unit resistances are solved from the electrical signals.

According to some embodiments of the present disclosure, the discrete film unit resistances are solved under constraints.

According to some embodiments of the present disclosure, the discrete film unit resistances are solved under smoothness constraints.

According to some embodiments of the present disclosure, the discrete film unit resistances are solved under length constraints.

According to some embodiments of the present disclosure, the sensing circuit is calibrated under known applied target physical quantities.

According to some embodiments of the present disclosure, the calibration determines values of the sensing circuit's reactive elements.

According to some embodiments of the present disclosure, the calibration determines resistance/physical quantity relation of the resistive film.

According to some embodiments of the present disclosure, parasitic effects such as temperature and strain are compensated in the measurements provided by the readout controller.

According to some embodiments of the present disclosure, parasitic effects are measured on a dedicated FPC layer of the sensing circuit, and subtracted from total measurements.

According to some embodiments of the present disclosure, the sensing circuit includes an isolated resistive wire.

According to some embodiments of the present disclosure, the wire is twisted to create a twisted pair between the two portions of the wire.

According to some embodiments of the present disclosure, the probe body diameter is smaller than 0.5 mm along the longitudinal extent of the sensing circuit.

According to some embodiments of the present disclosure, the probe body diameter is smaller than 0.36 mm along the longitudinal extent of the sensing circuit.

According to some embodiments of the present disclosure, the variably resistive film includes magnetoresistive film.

According to some embodiments of the present disclosure, the FPC is attached to an elongate device defining a lumen.

According to some embodiments of the present disclosure, the longitudinally extended device is a catheter.

According to some embodiments of the present disclosure, the FPC sensor is wrapped helically around a longitudinally extended device.

According to some embodiments of the present disclosure, the magnetoresistive film is divided into units, laid in alignment with one of more of the X, Y, Z and axes of the longitudinally extended device on each winding.

According to some embodiments of the present disclosure, the resistances are solved under sensor position smoothness and length constraints.

According to some embodiments of the present disclosure, the FPC sensor is calibrated using an EM model of transmitted fields.

According to some embodiments of the present disclosure, the readout controller determines the measurements using calibration-determined values of the sensor's reactive elements.

According to some embodiments of the present disclosure, the readout controller determines the measurements using a resistance/magnetic field relationship of the magnetoresistive film.

According to some embodiments of the present disclosure, the readout controller determines the measurements using a calibration-determined geometry of the FPC sensor.

According to some embodiments of the present disclosure, the magnetoresistive film is magnetized using high electrical current pulse through connections ports of the sensing circuit.

According to some embodiments of the present disclosure, the FPC sensing circuit configuration is duplicated in a double symmetrical FPC sensing circuit.

According to some embodiments of the present disclosure, the readout controller uses the symmetric configuration to determine a twist of the sensor.

According to some embodiments of the present disclosure, the readout controller uses the symmetric configuration to compensate for temperature and strain effects.

Unless otherwise defined, all technical and/or scientific terms used herein have the same meaning as commonly understood by one of ordinary skill in the art to which the present disclosure pertains. Although methods and materials similar or equivalent to those described herein can be used in the practice or testing of embodiments of the present disclosure, exemplary methods and/or materials are described below. In case of conflict, the patent specification, including definitions, will control. In addition, the materials, methods, and examples are illustrative only and are not intended to be necessarily limiting.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, microcode, etc.) or an embodiment combining software and hardware aspects that may all generally be referred to herein as a “circuit,” “module” or “system” (e.g., a method may be implemented using “computer circuitry”). Furthermore, some embodiments of the present disclosure may take the form of a computer program product embodied in one or more computer readable medium(s) having computer readable program code embodied thereon. Implementation of the method and/or system of some embodiments of the present disclosure can involve performing and/or completing selected tasks manually, automatically, or a combination thereof. Moreover, according to actual instrumentation and equipment of some embodiments of the method and/or system of the present disclosure, several selected tasks could be implemented by hardware, by software or by firmware and/or by a combination thereof, e.g., using an operating system.

For example, hardware for performing selected tasks according to some embodiments of the present disclosure could be implemented as a chip or a circuit. As software, selected tasks according to some embodiments of the present disclosure could be implemented as a plurality of software instructions being executed by a computer using any suitable operating system. In some embodiments of the present disclosure, one or more tasks performed in method and/or by system are performed by a data processor (also referred to herein as a “digital processor”, in reference to data processors which operate using groups of digital bits), such as a computing platform for executing a plurality of instructions. Instruction executing elements of the processor may comprise, for example, one or more microprocessor chips, ASICs, and/or FPGAs. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well. Any of these implementations are referred to herein more generally as instances of computer circuitry.

Any combination of one or more computer readable medium(s) may be utilized for some embodiments of the present disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device. A computer readable storage medium may also contain or store information for use by such a program, for example, data structured in the way it is recorded by the computer readable storage medium so that a computer program can access it as, for example, one or more tables, lists, arrays, data trees, and/or another data structure. Herein a computer readable storage medium which records data in a form retrievable as groups of digital bits is also referred to as a digital memory. It should be understood that a computer readable storage medium, in some embodiments, is optionally also used as a computer writable storage medium, in the case of a computer readable storage medium which is not read-only in nature, and/or in a read-only state.

Herein, a data processor is said to be “configured” to perform data processing actions insofar as it is coupled to a computer readable medium to receive instructions and/or data therefrom, process them, and/or store processing results in the same or another computer readable medium. The processing performed (optionally on the data) is specified by the instructions, with the effect that the processor operates according to the instructions. The act of processing may be referred to additionally or alternatively by one or more other terms; for example: comparing, estimating, determining, calculating, identifying, associating, storing, analyzing, selecting, and/or transforming. For example, in some embodiments, a digital processor receives instructions and data from a digital memory, processes the data according to the instructions, and/or stores processing results in the digital memory. In some embodiments, “providing” processing results comprises one or more of transmitting, storing and/or presenting processing results. Presenting optionally comprises showing on a display, indicating by sound, printing on a printout, or otherwise giving results in a form accessible to human sensory capabilities.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object oriented programming language such as Java, Smalltalk, C++ or the like and conventional procedural programming languages, such as the “C” programming language or similar programming languages. Additionally or alternatively, sequences of logical operations (optionally logical operations corresponding to computer instructions) may be embedded in the design of an ASIC and/or in the configuration of an FPGA device. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus such as an FPGA, or other devices such as ASICs to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such inspecting objects, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the present disclosure are herein described, by way of example only, with reference to the accompanying drawings. With specific reference now to the drawings in detail, it is stressed that the particulars shown are by way of example, and for purposes of illustrative discussion of embodiments of the present disclosure. In this regard, the description taken with the drawings makes apparent to those skilled in the art how embodiments of the present disclosure may be practiced.

In the drawings:

FIG. 1 schematically illustrates-wire sensor, with electrical properties divided to discrete units; either actually, or for purposes of description and analysis; according to some embodiments of the present disclosure;

FIG. 2 shows an example of a total impedance vs. frequency relation of a sensor with eight resistive elements and reactive components, according to some embodiments of the present disclosure;

FIG. 3 schematically illustrates-wire sensor, with electrical properties divided to discrete (but infinitesimal) units for purposes of description and analysis, according to some embodiments of the present disclosure;

FIG. 4 is a schematic flowchart describing a method of determining the position and shape of a flexible sensor within a set of generated magnetic fields, according to some embodiments of the present disclosure;

FIG. 5 schematically represents an endovascular tracking system, according to some embodiments of the present disclosure;

FIG. 6 schematically represents a two layer sensor FPC with discrete “barber pole” resistive elements, according to some embodiments of the present disclosure;

FIG. 7 schematically illustrates a twisted-pair variable resistive sensor configuration, according to some embodiments of the present disclosure;

FIG. 8 plots a magnetization to inductance relationship of an example magneto-inductive coil, wrapped around a high permeability non-linear magnetic core, according to some embodiments of the present disclosure;

FIG. 9 schematically represents an inductive sensor comprising discrete coils connected in series, according to some embodiments of the present disclosure;

FIG. 10 schematically represents a flexible sensor comprising a single coil made of discrete coil elements connected in series, according to some embodiments of the present disclosure;

FIG. 11 schematically represents a flexible sensor comprising a coil with continuously decreasing winding pitch, according to some embodiments of the present disclosure;

FIG. 12A plots a magnetization to inductance relationship of an example magneto-inductive coil with varying pitch, under an externally applied magnetic field which varies along the sensor's curve, according to some embodiments of the present disclosure;

FIG. 12B schematically indicates features of data acquired using a magneto-inductive sensor according to some embodiments of the present disclosure;

FIG. 13 schematically represents a guidewire having an integrated flexible sensor comprising a coil wound around a core which is constructed as a distal extension of the main guidewire body, according to some embodiments of the present disclosure; and

FIG. 14 schematically represents an endovascular tracking system, according to some embodiments of the present disclosure.

### DESCRIPTION OF SPECIFIC EMBODIMENTS OF THE INVENTION

The present invention, in some embodiments thereof, relates to the field of microprobe position and/or shape sensing and more particularly, but not exclusively, to sensing of probe positions and/or shapes using electrical field measurements.

### Overview

A broad aspect of some embodiments of the present disclosure relates to multiplexed, spatially resolved sensing of environmentally induced local effects upon a sensing device and/or material. In some embodiments, the sensing device and/or material is provided as a flexible and elongated sensor (i.e., long and thin). The linearly elongated sensor may be provided more particularly as part of (e.g., at a distal end of) a medical device probe, for example, a guide-wire, catheter-delivered tool, or catheter portion. The medical device probe may be configured for endoluminal navigation; e.g., for endobronchial and/or endovascular navigation, and/or specifically for neurovascular navigation and/or lymph system navigation.

In some embodiments, a purpose of the navigation is to bring and/or help guide a treatment, sampling, and/or diagnostic tool to a target of treatment, sampling, and/or diagnosis. In some embodiments, a purpose of the navigation is to reach another device such as a previously implanted device, for example to configure it, maintain it, and/or evaluate its functioning.

In some embodiments, an environmental parameter that is sensed by causing the local effects is local magnetic field strength and/or direction. Herein, these are also referred to together as the local magnetic field vector. Other definitions related to magnetic field properties are provided hereinbelow.

Spatially resolved measurement may rely, for example, on a sensing material having properties such as magnetoresistance (change in electrical resistance as a function of surrounding electromagnetic field influences), or on a sensing device comprising a coil having a core material with permeability that is non-linearly affected by magnetic fields it is exposed to. In some embodiments, environmentally-responsive inductance (non-linear permeability) and resistance properties are both provided over a spatial extent of a device, for example to provide separate measurement sources, to enhance each other's selectivity, and/or to assist calibration and/or compensation for secondary effects of the environment and/or intrinsic properties of the sensing circuit.

An aspect of some embodiments of the present disclosure relates to elongate probes which provide position sensing with respect to magnetic fields.

In some embodiments, a sensing region of the elongate probe is spanned by an electrical conductor (optionally a uniform wire or comprised a plurality of serially interconnected electrical conductors) which interconnects on first and second ends to a readout controller. The readout controller passes current into the electrical conductor, and measures electrical signals produced as a result. The signal itself integrates properties from all along the electrical conductor. Structurally, the sensing region is comprised of regions along its length which differ in their structure, and more particularly in an effect that this structure has on electrical current flowing through it, which is indicated in the measurements of the electrical signals. Furthermore, the sensing region is structured to produce this effect differently, depending on the magnetic field it is exposed to (e.g., local magnetic field strength).

The raw measurements from the electrical signals conflate these effects, producing total measurements, e.g., totaling the effects on the current produced everywhere along the electrical conductor.

In some embodiments, a processor decomposes the effects and assigns them, in a form such as an estimate of local magnetic field strength, to individual locations along the sensing probe. To do this, the processor uses the measurements themselves, along with models of the conditions under which the measurements were taken. In particular, expected responses to local magnetic field and the structural differences (at least their electrical effects) are modeled.

Furthermore, in some embodiments, a model of magnetic field distribution in a region containing the sensing region is used to determine where the sensing probe is, and/or its shape. For example, sensing region locations with their respective estimated local magnetic field strengths are assigned positions and/or a shape in 3-D spatial coordinates which consistently match the model of magnetic field distribution.

An aspect of some embodiments of the present disclosure relates to the use for shape determination and/or position finding of devices having electrical induction properties (e.g., core material electromagnetic field permeability) which experience spatially localized alterations induced by interactions with the environment. In some embodiments, the device is constructed so that the measurements can be spectrally multiplexed, allowing determination of which parts of the device are affected by external EM fields, and to what extent.

In some embodiments of the present disclosure, a sensor device comprises a continuous long and narrow inductive element (for example, 0.3 mm in diameter and 30 cm long coil). Alternatively, in some embodiments, a set of discrete inductive elements connected in series is provided.

However configured, inductor coils may be wrapped around a high permeability non-linear magnetic core; made, for example, of permalloy, supermalloy, mu-metal, cobalt alloy, or any other high permeability non-linear magnetic core. Relative permeability of a material is expressed as a dimensionless number. Permeability may be handled as complex number particularly in high-frequency applications. Examples of (real number) maximum relative permeability values for these materials include, for example: 100000 for permalloy, 800000 for supermalloy, 100000 for mu-metal, and 100000 for some cobalt-based alloys.

The non-linearity comprises changes in the permeability of the core material as it is exposed to electromagnetic fields having different properties (e.g., different intensities and/or frequencies). The core material may be intrinsically flexible; e.g., flexible when provided as a straight wire, as a coil, or as a braided or other compound structure. Additionally or alternatively, the extent of the core material may be given flexibility by coupling to and/or blending with another material. For example, in some embodiments core material is segmented, and the segments supported using another material such as a polymer coating or flexible strands (e.g., wires). In some embodiments, the high permeability non-linear magnetic core material is blended with a flexible matrix material. Thus configured, the flexible sensor is linear; that is, much longer than its diameter, and unbranched between two ends. The ratio of length to diameter is, for example, at least 20, and preferably at least a larger factor of 50 or 100. The maximum diameter, in some embodiments, is less than about 5 mm, and equal to or less than about 2 mm, about 1 mm, about 0.5 mm, or about 0.36 mm. In particular, reaching a diameter at least as small as 0.36 mm (0.014 inches) provides a potential advantage by being small enough for use as a neurovascular probe, and/or for use as a probe through lumens of the lymph system.

For use in shape determination and/or position finding, the sensor may be used alone, or coupled to another device; for example, embedded inside a catheter or an endoscope. In some embodiments, the sensor itself is further configured as a device with additional features as a use case may call for. For example, the core material may itself be formed to provide the lumenal wall of a microcatheter, and/or with the mechanical properties (e.g., steerability, torquability, and/or pushability) appropriate to a microcatheter or guidewire, or portion thereof. For example, it may be pushable, as the distal end of such a device, to a distance of at least 50 mm, into a body cavity region having an inner diameter of 5 mm or less. Optionally, at least a distal tip of the device is pushable into a body cavity region of at least 1 mm or less, 0.5 mm or less, or about 360 μm or less.

Suitable readout and processing of data measured from the sensor potentially provides real-time position and/or shape tracking of the catheter or other device so-equipped, for example, full shape tracking in 3-D. Except as otherwise indicated, “real-time” tracking comprises updating shape and/or position at least once per second; or more often, for example, a frequency of at least 5 Hz, 10 Hz, or 30 Hz. Updates may be used for system-internal purposes, e.g., in automated tracking, and/or may be provided as displays or other indications to device operators. Potential advantages of providing real-time tracking displays/indications to an operator include rapid feedback which allows adjusting control inputs to achieve intended movements of a device, and assisting in developing and/or maintaining for the operator a sense of the manipulated device as a “real” device, e.g., a unified impression of the state of the device, although based on indirect and/or artificially constructed measurements, and potentially measurements obtained through a plurality of indirect and/or artificial sensory modalities. Potentially, providing real-time feedback helps the operator learn to associate certain inputs to the device with certain likely results.

One manner of using a plurality of discrete coils is to connect wire pairs extending from coil ends to an external processing unit (readout controller) as multiple twisted pairs. The processing unit would measure the inductance of each discrete coil independently, for example using oscillation techniques or any other suitable method, to provide multiple inductance measurements which can be used as indications of the desired physical quantity or quantities. For example, the physical quantities may comprise magnetic field properties, and these may in turn be related to spatial positions having (or supposed to have) those particular magnetic field properties. However, in this configuration the number of wires may increase linearly with the number of coils along the sensor, reducing practicality for applications where small footprint is crucial.

In some embodiments of the present disclosure, an inductor comprising a single long coil (or set of coils in series) is provided, connected by its ends to an external processing unit. The processing unit measures total inductance of the inductor, which amounts to the line integral of the inductance density (i.e., the inductance per unit length) along the sensor's curve.

Under appropriate field conditions, the resulting single time-series measurement of total inductance along the sensor's curve, potentially contains information suitable for resolving information indicative of the sensor's shape and/or position. For example, in some embodiments, an EM field generator is provided which transmits a large number of spatially distinct AC fields, each at a different frequency. For example, 30 different fields may be transmitted through the vicinity of the sensor. The mean inductance measured using a single magneto-inductive coil can be used to extract 30 values from data sampled over a fairly short time period (for example, in a 30 millisecond window). Analysis for the extraction may, for example, use DFT (Discrete Fourier Transform) methods, correlation methods, or another suitable method. Together with knowledge and/or assumptions about the spatial distribution of the various EM fields being produced, these values are indicative of the sensor's full shape in space, relative to the transmitter. Under certain smoothness constraints, location constraints, distance constraints, and/or flexibility constraints applicable to the construction of the shape sensor, the sensor's full shape can be solved to a good accuracy based on the 30 sensed values. Herein, “smoothness constraints” refer constraints which encourage or require a model parameter representing a physical parameter such as magnetic field strength be continuously variable over space and/or time (i.e., not discontinuous). “Location constraints” refer to constraints on a model parameter representing sensed or a priori knowledge (complete and/or partial) of where an element is. For example, a sensor portion may be constrained to within a lumenal cavity of a body, e.g., insofar as it is known to have been introduced to that lumen, and assumed not to have punctured the lumenal cavity wall. “Distance constraints” refer to constraints on a model parameter representing a constant size or a size that changes within certain limits. For example, a known distance between two locations on a probe may be used as a distance constraint. “Flexibility constraints” refer to constraints on a model parameter representing limitations on how much a structure may bend. For example, a flexible probe may be modeled with an absolute limitation on its minimum radius of curvature, and/or curvature may be penalized within a certain range of curvature radii. Constraints may be implemented, e.g., in the form of absolute constraints, and/or in a form that affects likelihood. For example, they may be implemented in the form of penalties, such as penalties applied as errors during a computational process of error minimization to find a physical model state that explains sensed data.

Due to footprint constraints in several applications, specifically in medical applications, it is a potential advantage to connect as few wires as possible to the shape sensor. Since a shape sensor comprising a single magneto-inductive coil as described above may involve the use of an unusual and/or expensive and/or impractical EM field generator (which generates a large number of different alternating magnetic fields), it is a potential advantage to use a solution which: (a) can work with a simpler EM transmitter (for example, one only transmitting 3-6 distinguishable fields), and (b) works without using many wires connecting between the sensor and the external processing unit.

In some embodiments of the present disclosure, a shape sensor is provided which uses as few as 2 wires connecting it to an external processing unit, while providing multiple inductance measurements which can be referred to conditions (magnetic field state) at specific regions along the sensor's curve. This may substitute for the alternative of making discrete individual inductance measurements along the curve.

Not only is the inductor constructed in a spatially inhomogeneous manner (that is, it has different coil winding pitch or other construction parameter which produces different baseline inductance per unit length at different positions along it), but also its high permeability non-linear magnetic core varies additionally according to the local magnetic field vector it is exposed to.

Upon suitable calibration and biasing, this results in a device that has certain expected overall inductance (e.g., as measured by probing circuit resonance) within reference EM field conditions which changes to different values depending on how much current is passed through the inductor. Adjusting the current changes the inductance, because a change in current changes the applied magnetic field in the inductor—which in turn acts on its own (non-linearly responding) core to change its properties. There is thus a calibrated current-inductance relationship which is alternatively expressed as a magnetic field-inductance relationship.

This relationship changes when the device is exposed to new external EM field conditions, since these also shift the permeability of the non-linear magnetic core. This can be probed with the same range of currents to which the device was originally calibrated, e.g., using a time varying (for example, sinusoidal or other waveform) current. For example, a 1 MHz sinusoidal current is provided. In some embodiments, current is provided according to another repeating waveform shape; for example, a triangle wave, a sawtooth wave, or another continuously or non-continuously varying signal waveform. Optionally, the driving circuit provides current directly as a targeted waveform. Optionally, the driving circuit drives a voltage to a selected waveform, with the associated current following according to overall circuit characteristics, potentially including distortion of the voltage waveform.

The results measured are indicative of a new relationship between current (or imposed magnetic field) and inductance, shifted according to the influences of magnetic field strengths in the sensed environment. More particularly, magnetic field-induced shifts in magnetic core permeability, whatever they are, may be assumed to account for shifts in the relationship.

Thus, the sensor overall comprises sensor regions, each providing to the sensor a variable electrical inductance, characterized by a baseline partial inductance (e.g., the inductance of the region in the absence of magnetization), a sensing partial inductance, corresponding to the strength of externally imposed magnetization, and an internal partial inductance, corresponding to self-magnetization when a current flows through the sensor region. The sensing and internal partial inductances may alternatively be referred to as variations to the baseline inductance (which May be negative or positive in sign). The variations may be referred to as “responsive to”, e.g., externally imposed magnetization and currents flowing in the sensor. To facilitate analysis making use of not-discrete sensor regions, references to inductance may be replaced by references to (linear) inductance density, with integration, e.g., along coil lengths, being treated separately.

Mathematically, a set of shifts that could account for the change can be determined, for example using non-linear optimization approaches. Calculations for this, in some embodiments, make use of knowledge of how inductance density is (inhomogeneously) distributed along the sensing region of the probe, and/or the inductance curve of the core material (e.g., its change in permeability as a function of magnetization). With suitably different inductances along the probe, there may only be one set of shifts which is physically plausible.

In some embodiments, this may be described informally as follows. Since each suitably distinct (although perhaps overlapping) “element” along the probe has a different inductance density, it also imposes a different bias upon itself when operated at a given current—and a different current-to-inductance curve. The curves may be similar in shape for different elements, but shifted from one another. In operation, shifts also include effects that local differences in external magnetic field vector (e.g., strength and/or direction) impose. As far as inductance changes go, each individual element acts like it is seeing a different current. When the curves are, in effect, added together by a measurement that treats them as one larger inductor, the features defined individually blend together. Since the shifts of the curves under calibration conditions place them in distinct baseline offsets (e.g., peaks in different places, linear regions in different places, and so on), then additional offsets induced from external magnetic fields can be assigned to specific elements.

These considerations show how magnetic field vectors (and/or their strength) may be localized along a 1-D parametric space defined by the longitudinal extent of the probe. To convert this to the curvature of that probe (and its parametric space) in 3-D space, the probe's detected magnetic field vectors are mapped to known spatial distributions of magnetic field vectors. The shape and position of the probe are given by offsets, rotations, and contortions which preserve consistency between magnetic field strength values measured by the probe, and those known to exist in 3-D space. This process may be assisted by knowledge of the probe's actual length, and assuming plausible limits on how the probe can move and bend. More than one magnetic field may be used, e.g., 3-6 magnetic fields operated at different frequencies, allowing the separation of field influences, e.g., using discrete Fourier transform methods, correlation methods, or another frequency decomposition method. The magnetic fields are preferably arranged in a region of interest so that different regions have combinations of field vectors which are at least in part decorrelated, e.g. the magnetic fields have magnitude gradients directed to have mutually orthogonal components. In this way, each region is magnetically “tagged” to be measurably distinct from its neighbors in any direction, and preferably to be measurably distinct from all other regions which a portion of the probe might enter. Variously directed magnetic field magnitude gradients may be interpreted as establishing respective coordinate axes that have known and/or predictable relationships with 3-D position coordinate axes (and optionally also with up to 3 rotational coordinate axes). In some embodiments, measurements of magnetic field strengths in combination are mapped to position and/or orientation coordinates through this relationship, also referred to herein as a “mapping” of field strengths and/or directions to spatial coordinates. The mapping relationship may be only partially known, e.g., estimated from theoretical operating parameters, and potentially only partially calibrated. Optionally the mapping relationship used is corrected and/or constrained by other factors such as distances known to be fixed, angles known independently, measurements at particular known spatial positions, and/or other data.

Although having particular value for shape sensing, the same physical principles are useful, even to detect single sensor positions. For example, in some embodiments, a sensor comprises a single sensing element, e.g., a single short coil wrapped around a high permeability non-linear flexible magnetic core, such as a high permeability magnetic wire (e.g., made of supermalloy). Through the measurement of variations in its inductance, the sensor provides readings of magnetic fields at a single position and orientation in space, for example, at the tip of an endoluminal steerable medical device, where the coil is positioned. The device may be a guidewire, in which case electromechanical properties of the flexible magnetic core material may themselves provide suitable steerability, torqueability, pushability etc. The same material's highly magnetic permeability is what makes the coil magneto-inductive, that is, enables magnetic field measurements at the coil through measurements of magnetic-inductance non-linear relationship. Exploiting the device's core material both for mechanical requirements of the steerable device as well as for its magnetic properties has potential advantages for the construction of ultra-thin devices which can be EM-tracked in 3-D. In effect, a portion of the body of the device needed anyway for its mechanical functions is used also as part of a magnetic sensor.

An aspect of some embodiments of the present disclosure relates to the integration of magnetic sensors into the body of long, thin medical instruments such as guidewires.

Endovascular guidewires are typically long and thin (for example, a typical guidewire can be 180 cm long and have an outer diameter of 0.36 mm), and depend for their function on mechanical function aspects such as:
Pushability: the ability to accurately transfer axial motion at the proximal end to the distal end.
Torqueability: the ability to accurately transfer rotational motion at the proximal end to the distal end.
Trackability: the ability to follow a tortuous path defined by the anatomy or another device such as a catheter.
An atraumatic design: avoid applying high pressure on the surrounding tissue as to not cause damage.
Kink-resistance: the ability to bend in tight radii without buckling.
Durability: the ability to bend and flex multiple times without failing.

While functions like torqueability and pushability require high torsional and axial rigidity, other functions such as trackability and atraumatic design require low rigidity and a smooth transition in rigidity between different sections of the guidewire.

In some embodiments of the present disclosure, a core and coil design is provided in which the core is integrated into the guidewire along a portion of its body (i.e., in place of at least a portion of other support materials). This has potential advantages for addressing functional tradeoffs, as well as the functions of kink resistance and durability.

The core may be provided as a solid straight wire providing mechanical strength and stiffness. The coil portion wrapped around it may promote flexibility, durability, and kink-resistance. Desired mechanical properties are achieved, in some embodiments, by balancing the different functional parameters promoted by the core and coil, for example using design parameters like material selection, core diameter, coil wire diameter, and/or coil average winding diameter.

In some embodiments of the present disclosure, a high permeability non-linear magnetic material chosen as the core material, for example permalloy, has roughly similar mechanical properties to stainless steel, a material commonly used for guidewire cores. Using an isolated conductive material, parameters of the coil can be tuned cooperatively with parameters for shape sensing by using the methods described above.

Any parameter affecting the inductance of the coil can be varied to create a single coil with varying properties that allow shape sensing with as little as two wires as described above, for example the core diameter, the average diameter of the winding of the coil, or the thickness of the coil wire can be changed along the length of the guidewire. Each of these parameters can be controlled individually to create variations in coil geometries. It is noted in particular that variable pitch winding of the coil may be useful to provide a smooth transition of flexibility, e.g., from relatively stiff to relatively flexible behavior, which may help to reduce stress focusing which potentially occurs at sharp transitions between relatively compliant and relatively stiff device portions.

Optionally, magnetic properties of the materials used for coil and core are varied to achieve the same effect. This parameter may be used in probe design whether the probe is used as part of a guidewire body or otherwise provided. For example the core can be made from a high permeability non-linear magnetic material mixed with in varying ratio of another material to create varying magnetic properties along the core's length. For example, supermalloy is an alloy composed of nickel (75%), iron (20%), and molybdenum (5%). A modification of a custom supermalloy flexible wire can be used as the magnetic core for the sensor, where the composition of the alloy varies along the sensor's length. For example, at the sensor's tip the composition can be as described above. Then, towards the proximal part of the sensor the alloy can be modified continuously or discretely (for example, by joining multiple segments of different compositions) such that the amount of nickel decreases in favor of the amount of iron or the molybdenum, for example, the composition changes continuously from 75% nickel, 20% iron, 5% molybdenum to 50% nickel, 45% iron, 5% molybdenum. Alternatively, the amount of nickel increases while reducing iron and/or molybdenum; for example, the composition changes continuously from 75% nickel, 20% iron, 5% molybdenum to 95% nickel, 0% iron, 5% molybdenum, or in any other suitable manner.

Optionally, an additional material is added (e.g., to the alloy) to tune mechanical properties of the core along the sensor's length as well as its magnetic permeability curve. Modification of the alloy affects the magnetic permeability properties of the material. This can support the solvability of an equation system (e.g., by further distinguishing the weighting of different regions of the sensor probe in total measured inductances) which is processed using data from total inductance measurements to produce separated measurements of a plurality of magnetic field values along the sensor's length.

Accordingly, varying the alloy composition along the sensor length in turn controls magnetic permeability properties of the alloy along the sensor's length. This may be additional or alternative to changing the winding pitch of the coil wire around the magnetic core, or modifying the core's diameter along the sensor's length. The changing composition helps shift the inductance curves of sensor portions away from each other, to promote sensing a plurality of magnetic field values along the sensor's curve.

When combined, more particularly, in a guidewire, a probe portion of the guidewire (which optionally comprises as much of the guidewire as is considered desirable, including the whole guidewire) can be constructed which both has the desired mechanical properties required for various clinical uses as a guidewire, and can be fully shape tracked in 3-D in real-time.

Terms used herein to refer to properties of magnetic fields may be understood according to the following explanations and definitions.

Local magnetic field strength in its totality may alternatively be referred to as the magnetic field vector's magnitude, and direction may be referred to as that magnetic field vector's orientation. However, references to magnetic and/or magnetization field strength as such include the potential for signed values (negative or positive). This allows, for example, magnetic fields from a plurality of sources to potential adding up in opposite directions.

Where measurements of a magnetic field vector are potentially ambiguous as to a particular magnitude and orientation (e.g., because the sensor is anisotropically sensitive, measuring the vector anisotropically so as to indicate only a portion of its full magnitude), reference to the field's “strength” may be considered as a shorthand for what is measured, though this may in some embodiments be corrected to the entire local magnetic field vector by applying knowledge of the sensor's anisotropies and other available constraints. Reference to measurements of magnetic field, whether referred to as of strength, magnitude, vector, direction, or orientation, should be understood to refer to measurements of local magnetic field, e.g., its properties in a particular region of space to which the measurement is assigned. This may be, for example, a region including the position of the measurement device, or the position of a relevantly affected portion thereof. Inhomogeneities of magnetic field vector existing within that region are generally subsumed within a single measurement (e.g., averaged, according to whatever weighting the construction and sensitivity of the measurement device imposes). Reference to the strength or magnitude of a time-oscillating magnetic field vector may be understood as referring to its root mean square (RMS) strength/magnitude, also sometimes referred to as its DC strength/magnitude (or “value”, in whatever terms are set by the conditions of the measurement). Magnetic field amplitude refers to half peak-to-peak amplitude of a time-oscillating magnetic field vector (e.g., if non-rotating and centered on a magnitude of 0, half the sum of the peak magnitudes in either direction).

Furthermore, in relation to cases wherein a magnetic field is, more particularly, generating effects on the permeability of a material, it may be alternatively referred to herein for emphasis of this point as a “magnetization field”, associated in turn with properties such as “magnetization field strength”. However, uses of terminology based on “magnetic” should not be construed as thereby excluding magnetization effects. Also, the term “magnetization” by itself may be used to refer to magnetization field strength.

Permeability has units of inductance per unit distance (e.g., H/m in SI units). Along a certain axis, such as the longitudinal axis of an elongated sensor body, permeability may also be referred to as a measure of “inductance density”, or “linear inductance density”. Inductance density as such, however, may be the product of further parameters, such as coil geometry and current. Due to the close relationship between inductance and permeability changes, “inductance” may be referred to as changing when permeability changes, and vice versa.

Where the term “average” is used in reference to a measurement of time-oscillating magnetic field, root mean square averaging is assumed unless otherwise specified. In a strict sense, average magnetic field direction or orientation of a time-oscillating magnetic field may be null. Where this is a potential concern, reference to direction/orientation may be understood to refer to the orientation of an axis of oscillation of the instantaneous magnetic field magnitude. Unless otherwise specified, a reference to a time-varying magnetic field may be understood as emphasizing the case of a time-oscillating magnetic field (e.g., as a preferred embodiment).

An aspect of some embodiments of the present disclosure relates to spectrally multiplexed, spatially resolved sensing of environmentally induced local changes in the resistivity of a sensing material. In some embodiments, the sensing material is provided as part of a medical device probe, for example, a guide-wire, catheter-delivered tool, or catheter portion.

In some embodiments, an environmental parameter that is sensed by causing the resistivity changes is local magnetic field strength and/or direction. Additionally or alternatively, in some embodiments, another environmental parameter induces resistivity changes; for example, temperature. In some embodiments, the environment interacts with the sensing material to generate stress and/or strain in the sensing material, and the resistivity changes in response.

Certain materials are particularly susceptible to changes in resistivity in response to particular conditions, and/or commonly used as sensing material in applications that use these changes in resistivity. For example, permalloy (e.g., an alloy comprising about 80% iron and 20% nickel) is noted for having up to about a 5% variation in electrical resistance in response to an applied magnetic field. Furthermore, its magnetoresistance properties are anisotropic. This makes it useful for detecting both magnitude and direction of the local (intersecting) magnetic field. Thermistors (devices engineered for their relatively large and/or predictable resistivity changes as a function of temperature) are commonly produced using powdered metal oxides and/or silicon as their sensing material. The temperature coefficient of resistivity for material used in a thermistor may be, for example, about 5%. Piezoresistors display a change in resistivity as a function of strain; various semiconductor materials are commonly used as the sensing material in their commercial manufacture.

Sensing materials are available in different forms depending on their other material properties and available manufacturing technologies, for example continuously in the form of strips, tapes, wires, and/or sheets; and/or discretely, for example, in the form of sensor packages. Herein, the term “variably resistive sensing material” (or “sensing material”) refers to a material particularly selected in technological practice for its variable resistivity properties, i.e., its relatively large (in magnitude) coefficient of resistivity with respect to a change in a property such as local magnetic field, temperature, and/or strain.

In some embodiments of the present disclosure, a flexible resistive sensor is provided which measures a physical quantity (e.g., temperature, strain, magnetic field) along the full longitudinal extent (curve) of the sensor, optionally without using a plurality of discrete sensors along that curve. For example, in place of a plurality of separate films along the full of the sensor, the sensor comprises a continuous long film (for example, 1 mm wide and 30 cm long) or a set of discrete films connected in series; for example embedded on a flexible printed circuit (FPC) which gives the sensor shape flexibility. The sensor can be embedded in a device, for example, wrapped around a catheter. Suitable readout and processing of data measured from the sensor potentially provides real-time shape tracking of the catheter or other device so-equipped.

In the pursuit of miniaturization of sensing devices—in addition to the problems of miniaturization itself—there are potential drawbacks and/or difficulties arising in device aspects such as cost, manufacturability and/or reliability; particularly when a plurality of sensing locations are to be provided. There is also a problem of how these sensing locations may be addressed. In any of these cases, it is a potential advantage to reduce componentry, and/or to reduce numbers of individual connections.

In some embodiments of the present disclosure, a form of spectral multiplexing is used which allows a series-connected array of variably resistive sensing material portions to share electrical interfacing connections to a supporting measurement system in common. A potential advantage of such a structure is a reduced need for connections leading directly into the body of the series-connected array. Connections made from the ends, for example, can be used, in effect, to address sensing regions centered upon different locations all along the series-connected array.

In brief: in some embodiments, the impedance of the series connected array is measured at DC, and/or at one or more AC frequencies, for example, using a Wheatstone bridge. In some embodiments, using different measurement frequencies has the effect of integrating the resistivities of the sensing material portions with different respective weights (contributions) in terms of their effect on the signal response measurement. Additionally or alternatively, in some embodiments, the resistances of the series connected array are differentially sensitive to oscillating signals in the environment (e.g., because different resistive areas are exposed to different magnetic field oscillations, and so change their resistance with different timings).

In some embodiments, this is in part analogous to what happens when measuring the frequency dependence of the impedance of a transmission line. In the extreme of a DC (constant) driving signal, for example, all passive resistances are theoretically expected to contribute additively to the total resistance signal. As driving frequencies increase, however, circuit reactances (which, in the case of a simple transmission line, are understood to be present intrinsically) tend to shift this proportionality, e.g., to favor increased contributions from the near end(s) of the series-connected array.

Distinguishing, however, for some embodiments of the present disclosure, is that the “transmission line”—the series-connected array of variably resistive sensing material portions—is intentionally non-uniform. Moreover, as it is used for sensing, it is variably non-uniform: different portions change their resistivity differently depending on the local conditions that affect them.

There is, in short, information which, under suitable conditions, may be extracted to help characterize the present state of resistivities of different sensing material portions. Of course, the reactances themselves contribute to the frequency dependence of the measured signal, “contaminating” the information. However, with the exercise of suitable constraints and prior knowledge about the probe which comprises the sensing material portions, a computational process can potentially determine from this information a physically plausible model of the pattern of resistivity along the sensing material portions.

In some embodiments of the present disclosure, the sensing material portions are optionally discrete (e.g., individually packaged components) or continuous (e.g., in tape or strip form).

The concept of a “sensing region” is a model-dependent element distinct from the particular physical structure (e.g., discrete or continuous) of the sensing material. In some embodiments, there are identified within the series-connected array of sensing material portions a plurality of distinguishable sensing regions. The resistivity associated with a sensing region optionally is a single (e.g., weighted average) value, and the value is optionally associated with a particular (e.g., “center”) location along the array of sensing material portions. However, the collection of portions of sensing material contributing to sensing regions optionally cross physical boundaries that may exist between portions (e.g., they may include more than one discrete sensor). These collections optionally overlap with one another, wholly or in part. What distinguishes the locations of different sensing regions may be characterized as differences in the weighting which applies to contributions of the sensing material portions.

In some embodiments, and also in distinction to a normal transmission line, a series-connected array of variably resistive sensing material portions is embedded in a sensing circuit which is configured to enhance spectral impedance differences.

In a basic example, reactance may be controlled along the probe by adding small components (e.g., capacitors) at spaced intervals. The spaced intervals may be constant or non-uniform. Their capacitance values and/or the spaced intervals at which they are placed may be selected to differ from one another in relatively large steps. The capacitance values and/or their spaced intervals may be selected, for example, so that different portions of the probe (including different sensing material portions) are recruited into resonance at distinguishably different frequencies. These frequencies may be used in turn to determine which driving frequencies are used to operate the device for measurement.

In some embodiments, electrical properties of the sensing circuit are manipulated in another way which potentially increases the distinctiveness of different sensing regions for the de-multiplexing procedure. For example, insulating wire coating thicknesses may be varied, and/or conductor thickness itself may be varied. Optionally, arrangements of the sensing material itself may be changed. For example, the sensing material may be straight or coiled (optionally differently in different longitudinal locations along the probe), and if coiled, may be coiled at different pitches in different locations along the probe. “Coiled” includes one or both of helical coiling of a length of the sensing material by itself around and along an axis (e.g., as in a coil spring), and twisting of a length of the sensing material together with another conductor—for example, a return wire for the sensing circuit, and/or another length of sensing material. Twisting in particular may be used to create a twisted pair configuration of the sensing circuit, and the degree of twisting (e.g., how many twists per cm are provided) may itself manipulate inductance and/or capacitance, optionally without the addition of discrete inductor and/or capacitor components.

It should be noted, furthermore, that the arrangement of the sensing material may affect its sensitivity, and/or what types of forces it senses. For example, a coil of a strain-sensitive sensing material placed near the tip of a probe is potentially well suited to detect axially compressive forces, while a straight length of such material may be better suited to detecting bending forces. Anisotropic magnetoresistive material can be oriented to detect magnetic fields better from one direction than another. Where the magnetoresistive material is wound around a longitudinal axis of the probe (e.g., a wound strip), the directional sensitivity may vary as a function of distance along the probe. Winding such sensing material at different pitches at different longitudinal regions along the probe may also assist in enhancing regional distinguishability.

It should be understood that there are aspects of probe calibration which potentially affect the process of de-multiplexing (e.g., by providing constraints and/or baseline values for the device). In some embodiments, the probe and/or sensing circuit comprise additional elements which assist in probe calibration. For example, where resistivity of a first sensing material is sensitive primarily to magnetic fields, but secondarily to strain, a second sensing material may be provided which is sensitive primarily to strain, and secondarily (if, practically, at all) to magnetic fields. Responses of the second sensing material may, for example, be arranged to counteract those of the first sensing material (e.g., with an opposite sign), or the second sensing material may be provided in a second sensing circuit, and measurements from the second sensing circuit used to constrain the solution which yields resistivities along the sensing material of the first sensing circuit. Furthermore, wherever two different lengths of sensing material can be compared (e.g., because they are immediately adjacent longitudinally, or running longitudinally alongside each other), differences between them may be indicative of local conditions. For example, strain-sensitive sensing material on an inner radius of a bending probe may experience compression, while strain-sensitive sensing material on the outer radius may experience strain.

An aspect of some embodiments of the present disclosure relates to the use of spectrally multiplexed, spatially resolved readout of resistance changes in a magnetoresistive material for position finding.

In some embodiments of the present disclosure, not only is a physical property measured along a probe; but also, the probe extends along an at least partially unknown curve in space. Determination of that curve is constrained using the measurements of the physical property. In some embodiments, moreover, the physical quantity is itself specially arranged to increase the amount of positioning information available from the measurements.

In some embodiments, accordingly, an environmental parameter is manipulated in order to provide a spatial frame of reference that allows measurements associated to particular longitudinal positions along a sensor (e.g., a sensor constructed as just described) to also be associated with particular regions in space. In some embodiments, the manipulation comprises inducing EM (e.g., magnetic) fields throughout a region being navigated by a probe such that there are expected and generally distinguishable field properties measurable at each location within the region. This May involve inducing a plurality of EM fields, from different directions, and with different frequencies, phases, and/or duty cycles. The measurements from the probe may thus be multiplexed not only according to the readout signals generated, but also according to properties of the environment such as the local intensity of each of a plurality of magnetic fields distinguished also by their frequency and/or direction. In this approach, resistances are determinable as just described. The resistances in turn vary characteristically according to where in the complex superimposition of imposed magnetic fields they are. This information allows the position and shape of the sensor to be determined.

However, in some embodiments, spectral de-multiplexing of the probe impedance into an “intermediate” resistance mapping is not performed.

The total DC resistance of the sensing material amounts to the line integral of the resistance density (i.e., the resistance per unit length) along the sensor's longitudinal extent. This provides a single measurement of resistance along the sensor's whole longitudinal extent. In a simplified case, where only one part of the probe is affected by the imposed fields, knowing that this part interacts with a certain combination of electromagnetic field vectors provides the information that this part of it must be at a certain location “tagged” with that combination, providing position information in its own right. When effects on several parts are combined into one reading, however, the problem is made more complicated by the superimposition of effects, so that the problem becomes one of determining a position which would be consistent with the aggregate measured effect of the imposed fields. This does not necessarily have to proceed through a stage of isolating resistance changes as such in particular regions of the sensor.

For use in electromagnetic (EM) shape tracking, an EM fields generator which transmits a large number of different (that is, spatially distinct in shape) AC fields in different frequencies, (for example, 30 fields) is provided. A different total DC resistance (or optionally AC impedance) will be measured at each of the frequencies used. These total resistances can be distinguished by temporal frequency decomposition, for example, using DFT (Discrete Fourier Transform) methods, correlation methods, or any other suitable method. These values are indicative of the sensor's full shape in space, relative to the transmitter. Under certain smoothness assumptions of the shape sensor, the sensor's full shape can be solved to a good accuracy based on the various (for example, 30) sensed values.

For example, with just three orthogonal fields (and amplitudes, computed by analyzing temporal frequency decomposition of total resistances converted into magnetic fields) to work from, it would potentially be possible to assign an average center location to the probe, but not its full shape or orientation. With resistances to fourth and further fields added, however, only certain shapes and/or orientations of the probe will be consistent both with that average center, and with the additional measurements. The more fields used, the more constrained is the shape and position of the sensor. However, it may be understood that generating a sufficiently large number of AC fields is potentially cumbersome for a variety of reasons (e.g., space and setup considerations), so that it is a potential advantage to provide a solution which works with a smaller number of EM fields (e.g., three fields).

In some embodiments, the environmental parameter is more focally manipulated. For example, one or more energetic foci (optionally one or more moving or scanning foci) may be generated in the vicinity of the probe, and used to identify positions of any selected portion of the probe based on when, where, and to what extent resistive changes along the probe occur, relative to where the one or more energetic foci are created. The energetic foci may, for example, comprise regions of heating, generated, for example, using focused ultrasound, focused EM radiation, or another energy source.

Before explaining at least one embodiment of the present disclosure in detail, it is to be understood that the present disclosure is not necessarily limited in its application to the details of construction and the arrangement of the components and/or methods set forth in the following description and/or illustrated in the drawings. Features described in the current disclosure, including features of the invention, are capable of other embodiments or of being practiced or carried out in various ways.

### Spectrally Multiplexed Longitudinally Extended Sensor

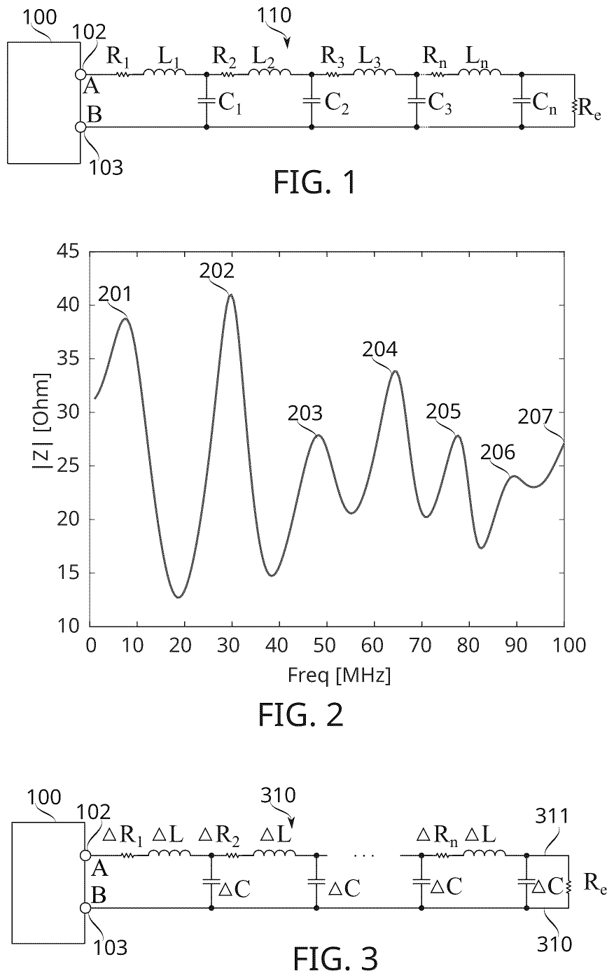

Reference is now made to FIG. 1, which schematically illustrates 2-lead sensor 110, with electrical properties divided to discrete units; either actually, or for purposes of description and analysis. The variable resistance of each sensing material portion is indicated by each resistance Rᵢ up to Rₙ. Reactive properties (Lᵢ, Cᵢ up to Lₙ and Cₙ) are configured to yield different total impedances under different test frequencies, in an overall configuration optionally similar to the electrical circuit topology of a transmission line. Sensor 110 is shown terminated with a termination Rₑ (a resistor, or another arrangement of terminating components). In some embodiments, sensor 110 comprises a flexible printed circuit (FPC).

In some embodiments of the present disclosure, sensor 110 comprises a long resistive film (or other form of sensing material), for example embedded as a layer of an FPC. At least for purposes of understanding and description, this sensing material can be considered as broken into discrete portions connected in series, each with its own resistance Rᵢ. This may correspond to an actual physical division, although the sensing material is optionally continuous. The sensing material can be connected to an external processing unit and readout controller 100) using, e.g., 30 two connection terminals 102, 103 (also labeled A, B).

Reactances of the circuit (due to capacitances Cᵢ and inductances Lᵢ, optionally of different values), may be (in whole or in part) intrinsic to the arrangement and physical properties of the resistive film and/or wires interconnecting portions thereof. Physically, reactances and/or resistances may be distributed continuously, but this can be represented for analysis to a selected accuracy by dividing the continuous distribution into any number of sensing units required (continuously distributed properties are discussed further in relation to FIG. 3). Optionally, the reactances are at least in part provided by added components; for example 0201 (0603 metric) SMD (Surface Mount Device) components.

The resistances Rᵢ (resistance of the i^{th} circuit unit, including resistance of a portion of the sensing material) are unknown, at least in part. In particular, at least the changes from some baseline due to environmental influences one the resistance of the sensing material are unknown. Determining these changes is of specific interest in some embodiments of the present disclosure, in order to extract localized sensing information about the environment. In order to produce multiple and differentially informative impedance measurements of the sensing material which may allow Rᵢ to be inferred, a multispectral approach is used. The processing unit 100 measures the complex impedance (resistance and reactance) of the sensor 110 at multiple frequencies; for example, from 1 MHz to 100 MHz. The measurements can be obtained, for example using an AC Wheatstone bridge. Optionally, measurements are repeated, for example, at a rate of about 1 kHz, or a higher or lower frequency.

Due to the reactances being physically associated to different locations relative to the sensing material portions of the circuit units, the unknown variable resistances of sensor 110 are combined differently (e.g., with different weights) for each test frequency. This produces a system of non-linear equations on the unknown Rᵢ elements, with known coefficients determined by the known values of the reactances and the test frequency. The system of equations can be solved, for example, using least squares optimization methods, permitting the values of the Rᵢ elements to be recovered.

More formally: denote by Rᵢ the unknown variable resistance of each i^{th} resistive film unit or other resistive sensing material portion of sensor 110. The resistive sensing material may be, for example, magnetoresistive, in which case its resistance in some location is indicative of the magnetic field at that location. Optionally or alternatively, the sensing material is thermo-resistive or of any other variably resistive nature. Lᵢ and Cᵢ are the known (and optionally assumed constant) inductance and capacitance values respectively distributed along the sensor; for example, arranged as depicted in FIG. 1.

Even if not actually constant, they may still optionally be treated as such, with an effects of their variation being collected, e.g., into the estimated varying values of Rᵢ. Assuming that such variation (if any) is at least correlated with variation in Rᵢ, error this may create can be corrected by suitable calibration. For example, an externally applied magnetic field may affect the inductance of some magnetoinductive films or nano coils. In any such case, demultiplexing methods described herein are adaptable as necessary to measuring the general varying complex impedance of films connected in series along a sensor. The methods are by no means restricted to measuring just the real-valued resistance of a plurality of discrete films or a continuous film as described above. In general, Rᵢ and R(x) can be thought of as complex numbers representing the varying complex impedance of a film and they can be measured using the exact same methods as were described.

Optionally, the circuit contains a termination resistor Rₑ, chosen, for example, according to the classical formula

$${R}_{e} = {Z}_{0} = \sqrt{\frac{L}{C}}$$

to match a (resistance-neglecting) characteristic impedance of the sensor, considered as a transmission line. This can help prevent measurement confusion due to signal reflections from the distal end of sensor 110. Where resistances introduce significant frequency dependence of the characteristic impedance, they are optionally also taken into account. Optionally, termination comprises additional passive components to better match terminal impedance through a wider range of the spectrum of driving frequencies used.

Sensor 110, in some embodiments, comprises an FPC with two lead terminals 102, 103. The lead terminals connect to an external processing unit 100. If the circuit were without reactances, then the measured resistance would be the sum of all Rᵢ's plus Rₑ, regardless of the impedance test frequency. This would preclude spectral de-multiplexing. However, since there is reactance, the total complex impedance between terminals 102, 103, denoted by Zₜₒₜ is frequency dependent. Zₜₒₜ can therefore be written as a function:

Zₜₒₜ=Zₜₒₜ(ω,R₁,R₂, . . . ,R_{N})

Where ω is 2πf and f is the test frequency. For any choice of w and resistor values Rᵢ the total impedance Zₜₒₜ can be computed by electrical simulation (for example, utilizing Ohm's law and Kirchhoff's circuit law). Insofar as the values of the reactive components inside the sensor are known, the total impedance can be estimated for any choice of values. The external processing unit measures Zₜₒₜ at multiple frequencies, for example from 1 MHz to 100 MHz, resulting in M (M in a range of 10-100, for example) measurements Zⱼ at frequencies ωⱼ. For each measurement j between 1 and M, the measurements satisfy:

Zⱼ=Zₜₒₜ(ωⱼ,R₁,R₂, . . . ,R_{N})

Where ωⱼ is the known test frequency of the j^{th} measurement and Rᵢ are the unknown variable resistances of the i^{th} resistive element (e.g., film unit). Each of the M measurements contributes an equation (usually non-linear) on N unknown variables Rᵢ, for a total of M equations on N variables. If M≥N and if the system of equations is regular enough (which depends on the choices of Lᵢ, Cᵢ) then the system is solvable using non-linear methods and the Rᵢ solution is unique.

### Resolving Different Longitudinal Positions

Reference is now made to FIG. 2, which shows an example of a total impedance vs. frequency relation of a sensor with eight resistive elements and reactive components, according to some embodiments of the present disclosure. Optionally, the resistive elements comprise a single continuous film of sensing material, or serially-interconnected segments of sensing material (e.g., film units). The reactive components are provided as discrete components in an arrangement embodying the circuit structure of FIG. 1. In this example, the capacitors used are 150 pF and the inductors used are 70 nH. A termination resistor is chosen with Rₑ=21.6Ω. These values are chosen in advance. The total impedance vs. frequency profile shown is for eight film resistance values Rᵢ: [3,1,4,1,5,9,2,6]Ω (while these happen to be initial digits of pi, they are selected merely as examples). The whole device is optionally constructed as an FPC.

Frequency response of the circuit is tested for a plurality of frequencies in the range of 1 MHz to 100 MHz. The frequency dependence of impedance is shown continuously, e.g., interpolated between measurements. With appropriately selected values, simulations predict that a plurality of distinguishable peaks arise in the graph of the sensor's frequency-dependent impedance. Different resistive elements (and changes in their resistance) contribute differentially to determining the magnitude of each peak.

Peak impedance values are not, in general, solely determined by the closest resistance value, but insofar as the weighting of resistance contributions changes from frequency to frequency, information about what pattern of resistances is consistent with the observed measurements is embedded in their values. It should understood that it is not necessary to confine analysis to the use of peaks, but these are notable for their visual correlation in amplitude with corresponding resistances.

Adding inductances as discrete components at discrete locations potentially helps emphasize the distinct contributes of resistive regions between them. However, this is not an absolute necessity, depending on noise conditions, the relative size of resistive changes, and other factors influencing signal to noise.

In order to further assist the solver, additional constraints can be added. Optionally, these relate to plausible constraints on the behavior of the physical quantity being measured. For example, with a longitudinally extended temperature sensor, the temperature can be assumed to be smooth along the curve to a certain extent (e.g., not varying more than a certain amount within a certain distance, and/or having a gradient not varying more than a certain amount within a certain distance). Time-dependent behavior is also an optional constraint; e.g., various forms of the assumption that moment-to-moment differences remain within some restrictive bounds.

Since the values of Rᵢ are solved using non-linear optimization methods, additional constraints may be introduced into the non-linear solver, in various ways; for example, by adding a weighted smoothness error term |Rᵢ−Rᵢ₊₁| in the total energy function being minimized.

A similar approach may be used with magnetoresistive sensing, exploiting contiguity and/or limits on rates of change of the magnetic field along the sensor's longitudinal extent.

In some embodiments, the total impedance of the circuit is measured for each frequency separately (e.g., at different times, driving the circuit with a different single frequency). Optionally, the impedance of a plurality of frequencies (e.g., all tested frequencies or any portion thereof) is measured in a single test. For example, a 1 MHz square voltage signal can be fed into the circuit. A 1 MHz square voltage signal contains a superposition of a 1 MHz sine/cosine wave and its harmonics, for example between 1 MHz and 100 MHz. By analyzing the circuit's 1 MHz periodic current function, the total impedance of the circuit for each frequency within the frequency range can be calculated. Optionally, another superposition of a plurality of frequencies is used.

In some embodiments, in contrast to direct embedding a sensing material in a single strip (e.g., a resistive film) along the longitudinal extent of an FPC, the sensing material is embedded inside discrete elements, for example, as a 0201 SMD component, similar to other standard passive electrical components. The 0201 SMD resistive component is special in the sense that it its resistance is largely due to a material selected because its resistance undergoes relatively large and predictable changes under certain physical conditions. For example, a magnetoresistive element is selected which changes its resistance by up to several percent (e.g., 3%, 4%, 5%, or more), depending on the external magnetic field inside which it is located. Placing the resistive film inside a small discrete element is potentially advantageous since it concentrates the resistive film at a certain focal location along a sensor (due to its small size).

### Continuous Spectrally Multiplexed Longitudinally Extended Sensor

Reference is now made to FIG. 3, which schematically illustrates 2-wire sensor 310, with electrical properties divided to discrete (but infinitesimal) units for purposes of description and analysis. Variable differential resistances of the sensing material (provided, e.g., as a continuous film unit) to be measured are indicated by ΔRᵢ. Virtual differential reactive elements are indicated by ΔC and ΔL. The sensor is shown terminated with a termination resistor Rₑ.

The use of the δ prefixes indicates an emphasis in FIG. 3 on the continuously sub-dividable arrangement of electrical properties in some embodiments of the present disclosure. In relation, for example, to FIG. 1, it is described that the physical construction of the sensing circuit need not physically package resistances and reactances into discrete units. This construction may be used in all or some portions of the sensor. Accordingly, spectrally multiplexed operation of the sensing circuit may rely on inherent reactance. Potentially, this allows the sensor to be produced with a reduced maximum diameter, and/or reduced diameter in one or more particularly size-sensitive areas, for example, a reduced diameter throughout a distal tip region of the sensor.

In some embodiments, the intrinsic reactances are substantially the same at each unit, with only the resistances Rᵢ varying. The inductance and capacitance per unit length can be estimated for example using microstrip impedance calculators.

In some embodiments, intrinsic reactances are deliberately varied along the longitudinal extent of the sensor (in which case ΔC and ΔL are replaced by ΔCᵢ and ΔLᵢ). Variation is be implemented, for example, by one or more of: the twist density (turns per unit length) of the wires, electrical insulation properties (e.g., insulating lacquer thickness), and/or conductor shape (e.g., tapering width, thickness and/or diameter; and/or longitudinally differentiated flattening of wires).

In some embodiments, the sensing material of sensor 310 comprises a thin, long resistive film located along resistive pathway 311 (e.g., interconnecting terminal 102 and terminating resistor Rₑ), optionally as a component of an FPC. Optionally, the film comprises a continuous extent comprising all of resistances Rᵢ, effectively arranged in electrical series. An electrical return path 310 (e.g., interconnecting Rₑ and terminal 103) is provided, for example, as a second wire and/or as a second layer of the FPC. Optional termination resistor Rₑ (optionally together with other components) is selected to match the characteristic impedance of the sensor.

As before, the total impedance of the sensor is measured through a frequency range, for example, between 1 MHz and 100 MHz. The sensor's sensing material (e.g., a resistive film) has a resistance per unit length that varies according to a magnitude, direction, or other quantity measuring a certain physical phenomenon.

For example, a continuous magnetoresistive film has a varying resistance per unit length along that film, the variation being an effect produced by a non-uniform magnetic field inside which the sensor is located. The sensor's self-inductance and self-capacitance are responsible for producing different impedance readings at each test frequency, and their differences are indicative of the continuous (but varying) resistance per unit length of the film, R(x):

Zⱼ=Zₜₒₜ(ωⱼ,R(x))

In the above, Zⱼ is a function of the test frequency and the full resistance per unit length of the film, R(x), wherein x is the position along the film. Insofar as a finite number M of test frequencies is used for measuring the impedance, R(x) as defined cannot be solved to an arbitrary resolution; however, resolution constraints and/or other constraints/assumptions may be posed that allow finding potentially useful solutions. For example, R(x) can be assumed to vary smoothly along the film (for example, based on the assumption that the physical quantity being measured is smoothly varying in space and that the sensor's own shape is likewise smooth). The criteria for “vary smoothly” may be selected appropriately to conditions; for example, to exclude discontinuities above a certain magnitude in value and/or rate of change in value. Smoothness optionally imposes filtering constraints limiting the frequency-dependent amplitude of changes. Time-based constraints may be applied, e.g., to ensure that measurements near each other in time have correspondingly similar solutions. An earlier-determined solution may also provide an advantageous starting point for use with later-acquired measurements.

In more particular examples: R(x) is optionally modeled as a polynomial of finite degree N, or as a spline between N control points. These embodiments re-cast R(x) as a function of N variables Rᵢ. Again, Zⱼ can be represented using relations of the N “discrete” Rᵢ's.

Position Finding with a Magnetoresistive Sensor

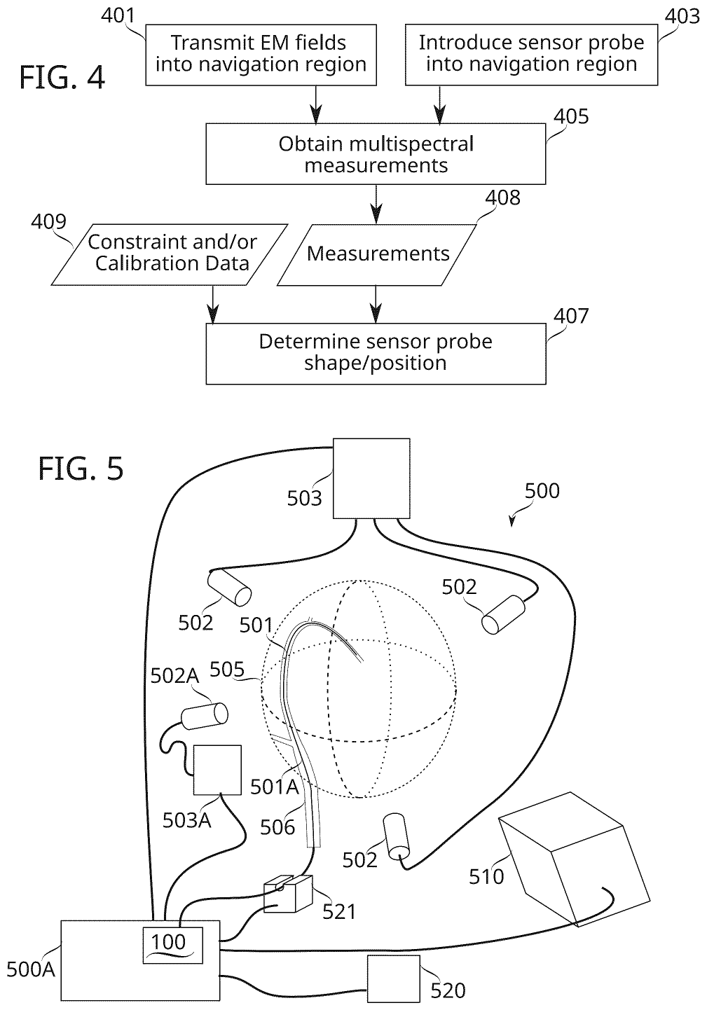

Reference is now made to FIG. 4, which is a schematic flowchart describing a method of determining the position and shape of a flexible sensor within a set of generated magnetic fields, according to some embodiments of the present disclosure.

At block 401, in some embodiments, an EM transmitter is activated to transmit a well-characterized set of electromagnetic fields into a region, for example, a tracking region of a living body within which the position of a probe is to be determined and/or tracked. The electromagnetic fields are “well-characterized” in the sense that magnetic field vector and/or frequency is predictable for positions within the tracking region. To at least a first approximation, these properties can be determined on a theoretical (e.g., simulated) basis based on the known positions 30 of field-generating magnets and their operating parameters. Optionally, calibration of any suitable type is performed to account for influences on the field due to other aspects of the environment. The aspects optionally include, for example, metals in known positions relative to the transmitter and/or sensor, properties of the tissue of the living body, stray magnetic fields, other nearby objects.

At block 403, in some embodiments, a probe comprising a longitudinally extended sensor comprising a magnetoresistive sensing material is introduced into the region of the living body through which the electromagnetic fields extend. Blocks 401 and 403 may be initiated in any suitable order. The introduction may be, e.g., as a guidewire leading a catheter, as part of a catheter, or as part of a longitudinally extended microsurgical tool of another type.

At block 405, in some embodiments, multispectral measurements are obtained using the sensor probe. The EM fields transmitted into the navigation region in themselves create a multispectral condition. The measurements optionally comprise readout of the resistance changes they create at any single suitable frequency. Optionally readout uses a DC signal. Alternatively, in some embodiments, readout comprises sequential and/or simultaneous excitements of the sensor circuit with a plurality of readout frequencies (e.g., 10, 20, 30, 40 or more frequencies) from within the range, for example of 1-100 MHz; for example as described in relation to FIG. 1. Optionally, readout frequency signal and EM field frequency signals are coordinated (e.g., synchronized to known phases).

Measurements are provided at block 408.

At block 407, in some embodiments, the measurements 408 and other information (e.g., constraint and/or calibration data 409) are used to find the sensor's curved position (and shape) in space. By the term “curved position” is meant a collection of positions along the longitudinal extent of the sensor, which together specify a shape of the sensor (potentially and likely curved in some degree), as well as an orientation and overall position (e.g., mid-point position) of that shape. The shape and position may be constrained, for example, by an anatomical track the sensor follows through the region of the living body, e.g., a track defined by vasculature, a portion of the gastrointestinal tract, and/or spaces between organs.

As before, total impedance of the sensor is measured at a frequency range, for example, between 1 MHz and 100 MHz. The sensor's resistive film has a varying resistance per unit length, due to the varying effect of the external physical quantity applied on that film. For example, a long magnetoresistive continuous film has a varying resistance per unit length along that film due to the effect of an external non-uniform magnetic field inside which the sensor is located. As before, the sensor's self-inductance and self-capacitance are responsible for producing different impedance reading per each test frequency, which are indicative of the now continuous resistance per unit length of the film, R(x):

Zⱼ=Zₜₒₜ(ωⱼ,R(x))

That is, Zⱼ is now a function of the test frequency and the full resistance per unit length of the film, R(x) where x is the position along the film. Since possibly only a finite number M of test frequencies is being used for measuring the impedance, additional constraints are posed to allow solving R(x).

For example, R(x) is assumed to be smooth along the film (for example, based on the assumption that the physical quantity being measured is smooth in space and that the sensor's curve is smooth). In this case, R(x) is optionally modeled, for example, as a polynomial of finite degree N, or as a spline between N control points. In the latter case, R(x) is again a function of N variables Rᵢ, and Zⱼ can again be represented using relations of the N “discrete” Rᵢ's.

Instead of posing just a smoothness constraint on the physical quantity along the sensor's curve, further constraints may be used which are stricter and can therefore aid the solver converge faster and to a more accurate result. For example, a magnetoresistive curve sensor is usually placed inside known magnetic fields generated by a controlled EM fields generator. In this case, the magnetic field at each point in space is known exactly at each moment in time. Synchronizing between the transmitter and the curve sensor, the magnetic field is known at any position along the sensor's curve at any moment, assuming that the sensor's curve position in space is known. Rather than explicitly searching for R(x) that yields the impedance measurements Zⱼ, the problem then becomes finding the sensor's curve position in space at any point in time, such that the known magnetic field along that curve yields the observations Zⱼ. Formally, Zⱼ is written as a function of the sensor's position:

Zⱼ=Zₜₒₜ(ωⱼ,r(x))

Where r(x) is the position of the sensor curve in 3-dimensional space, relative to the transmitter. As before, r(x) can be assumed to be smooth and can be described with N discrete positions rᵢ, between which a spline is fitted. It can also be assumed to have a fixed and known length at any point in time. Finding the curve's position in space doesn't necessarily allow for an arbitrary resistance function R(x) to be solved, but the function may be soluble for a continuous resistance which is observable inside the known transmitted field and under smoothness constraints of the sensor curve. This puts a stronger constraint on the solved resistance function R(x), which improves the solver's speed and accuracy.

By whichever method is used, task of finding solutions is aided by numerous available or potentially available constraints, represented in aggregated by block 409. Such constraints optionally include, for example, any of: limitations on the plausible minimum radius of curvature of the sensor, the fixed and known distance along the sensor between each location x, previous known positions of the sensor in space, limits on how the sensor can move along its track (e.g., advancing and withdrawing along its already established path of introduction), and/or prior anatomical knowledge (e.g., a previous static image of the anatomy along which the device is navigating).

### Calibration

Solving for Rᵢ from the observations Zⱼ makes use of the idea that all the other characteristics of the circuit are known, or at least that discrepancies can be ignored. For a discrete-component sensor this means that Lᵢ, Cᵢ are known. For a continuous sensor this means that the impedance and capacitance per unit length (and so also the characteristic impedance) are known. If by some manipulation these also vary per unit length, then the function by which they vary is also known.

In reality, electrical components have tolerances, and potentially even environmental sensitivities of their own, so that their exact impedances may not be precisely known simply based on their specified values. Also (particularly in the case of discrete-component sensor embodiments) self-capacitance and self-inductance of the circuit may add “virtual” Lᵢ, Cᵢ. A portion of these variables may be accounted for through calibration (e.g., empirical investigation to detect and/or characterize them).

As an example of calibration: for a magnetoresistive sensor, a constrained calibration process is used in some embodiments, based on methods similar to those described above.

The sensor may be placed within a known transmitted magnetic field and its shape and position may be deformed by an operator, optionally in an unsupervised manner. Measurements in the no-field case may also be performed. At each moment in time, Zⱼ are sampled, and these measurements are indicative of the Rᵢ's. However, more generally, Zⱼ are also indicative of the other circuit's characteristics: Lᵢ, Cᵢ. Under the constraints of a known transmitted magnetic field, plus curve smoothness and/or distance constraints, all of Rᵢ, Lᵢ, Cᵢ can be simultaneously solved in an optimization process (and not just Rᵢ as before). During calibration, there is potentially a lowered requirement for real-time performance, so the calibration solver can optionally configured to run to a higher level of convergence, and/or using a reduced number of constraints and/or approximations.

This calibration process may be performed for each sensor, e.g., factory performed as part of their production. As an optional constraint on calibration calculations, Lᵢ, Cᵢ may be assumed to have fixed values for all the measurements over time, while Rᵢ changes per timestamp (since the sensor moves). For the correct values of Lᵢ and Cᵢ, substantially all the constraints will be met over substantially all timestamps (within the limitations of sampling error and other noise): that is, that the sensed magnetic fields (derived from Rᵢ) will conform to the known transmitter field, and the solved sensor curve positions (derived from the magnetic fields) will be smooth. In addition, computing the desired physical quantity from resistance measurements Rᵢ relies on accurate translation between resistance measurements and the desired physical quantity (e.g., magnetic field, temperature). Resistance-to-physical quantity curves for each resistance Rᵢ may be calibrated as part of the same process.

In an alternative calibration process, a more supervised method can be used. In case of a sensor using magnetoresistive sensing material, the sensor is placed to extend through positions in space whereat the magnetic field is fully known. Possible values of Lᵢ, Cᵢ are then searched so that the solved magnetic fields will equal the target fields at these positions. For a thermo-resistive shape sensor, the sensor can be placed inside a special device which sets known temperatures at known positions along the sensor's curve. This provides the target measurements for the sensor. The circuit characteristics (i.e., Lᵢ, Cᵢ) can then be searched such that the finally computed Rᵢ will yield the target temperatures.

It may be noted also that providing certain combinations of sensing material—or even the same material in different orientations—may be of benefit in isolating desired sensing responses, compared to other resistive change which may occur.

For example, magnetoresistive materials may be anisotropic—responding more to fields oriented in one direction than in another. This could lead to “dead zones” for certain fields in certain locations where the orientation of the sensing material leads to insensitivity. In some embodiments, the sensing material is wrapped or twisted around the longitudinal extent of the sensor, and/or twisted on itself, and conditions and/or processing of measurements chosen in such a way that measured resistances are binned to combine sensing material in different orientations. Additionally or alternatively, local differences in material orientation are used to inform the system about sensor state. For example, as the sensor becomes twisted, two nearby portions of anisotropic magnetoresistive material may move relatively into or out of alignment in their orientations. Changes in the relative directionality of the magnetic fields they are sensitive to thereby becomes indicative of twisting of the device.

In another example, temperature response may be the same no matter what the orientation of a sensing material is, but strain sensitivity may be anisotropic. This may allow parasitic temperature responses to be isolated from strain responses, and potentially even allow even the same sensing material to be used for distinguishably measuring two or more environmental parameters.

### Endoluminal Navigation-Related Features and Applications

Reference is now made to FIG. 5, which schematically represents an endovascular tracking system 500, according to some embodiments of the present disclosure.

Sensor 501 is a spectrally multiplexed, longitudinally extended variable resistivity sensor, corresponding, for example, to one or more of the embodiments of FIGS. 1, 3, 6, 7, or another spectrally multiplexed, variable resistivity sensor. Sensor 501 is interconnected with external processing unit 500A and/or external processing unit 100 (e.g., via two or more connection terminals 102, 103, not shown). External processing unit 500A may fully incorporate external processing unit 100 (including, in particular, its function as a readout controller), and optionally provides other processing features, for example as described in relation to embodiments corresponding to FIG. 5, or other embodiments herein. In some embodiments, the functionality of a readout controller is provided separately from most or all data processing functions, i.e., separate from external processing unit 500A, but in communication with it so as to transmit measured data, and optionally to receive commands governing how sensor 501 is controlled and/or how measurements are collected from it. Readout controller functions include at least driving sensor 501 with electrical current used in magnetic field sensing. Moreover, readout controller 500B senses the magnetic field-reactive state of sensor 141, via the influence of its (variable) resistance on properties of the electrical current (for example, its oscillation frequency and/or amplitude of oscillation at one or more frequencies). External processing unit 500A may incorporate additional processing features. For example, it may perform integration of measurements made using sensor 501 with sensor data from another modality such as imaging device 510, or any additional sensors which may be provided as part of navigable probe 501A. In some embodiments, processing unit 500A processes according to inputs received from user interface 520 (e.g., according to selected views and/or operational modes), and/or performs generation of displays for user interface 520.

It should be understood that features described in relation to the system of FIG. 5 which are not directly dependent on magnetoresistivity per se are optionally provided in systems using another form of shape tracking sensor, for example, a magneto-inductive sensor, e.g., as described in relation to the embodiments of FIGS. 9-11. Similarly, features of FIG. 5 are combined freely in some embodiments with features of systems according to FIG. 14, insofar as they are compatible; e.g., insofar as they do not inherently depend on a particular sensor type (e.g., operating principle). In some embodiments, different devices having different sensor types (e.g., magnetoresistive and magneto-inductive) are used within a same system provided with features described in relation to any of FIGS. 5 and 14.

In some embodiments, a single device may be itself provided with a plurality of curve-sensing types (e.g., both magnetoresistive and magneto-inductive); along different regions of its length and/or along shared portions of its length. The extra data provides potential advantages, for example, in assisting the discovery of shape solutions, albeit potentially at the cost of complexity and/or device size. A magneto-inductive sensor is optionally provided together on a device with an additional sensor type described here particularly in relation to combination with magnetoresistive sensing; for example, one or more piezoelectric and/or thermoresistive sensors. Similarly, modes of navigation and/or navigation assistance described with respect to these additional sensor types are optionally provided together with systems providing magneto-inductive sensing for navigation.

Also provided, in some embodiments, are EM field control unit 503, interconnected with a plurality of field generators 502 (e.g., magnetic field generators). Field generators 502 are arranged around navigational region of interest 505, comprising, for example, a portion of a human body (e.g., a cranium).

Sensor 501, as part of a navigable probe 501A, is shown introduced into navigational region of interest 505, via a vasculature 506, of which a restricted portion is shown for purposes of illustration.

Among the potential advantages of a variable resistance-based sensor 501 with spectrally multiplexed readout is its potentially very small cross-section; potentially as small as can be realized by a pair of wire conductors. In some embodiments, the sensor 501 is suitable for use in a probe 501A with a maximum cross-sectional diameter of 1 mm or less, 750 μm or less, 500 μm or less, 400 μm or less, 350 μm or less, 250 μm or less, or less than another cross-sectional diameter. The probe may be integrated into a guidewire, or other longitudinally extended tool suitable for insertion into longitudinally navigated anatomical channels such as blood vessels and/or airways. Additionally, the probe can serve as a guidewire, sharing both electromagnetic and mechanical properties (e.g., pushability, torqueability etc.) needed for a guidewire, while being a magnetoresistive or magneto-inductive sensor as described herein.

Another potential benefit of such sensors, particularly but not exclusively in a twisted wire pair configuration is design simplicity and cost, with corresponding potential advantages for use as a disposable device. For example, in certain applications a plurality of magnetoresistive fully tracked “guidewires” can be introduced to a patient's organ and provide a fully tracked real-time skeleton of that organ for certain uses, such as modeling the deformation of that organ during a medical procedure.

The most distal portions of the sensor 501 are optionally constructed thinner than more proximal portions. This allows tradeoffs, e.g., such as adding discrete components in larger-diameter regions, further differentiating them electrically from the electrical properties of smaller-diameter regions where volume to add discrete components is more restricted, or absent.

Insofar as it is common for guidewires to comprise metal portions to achieve appropriate levels of stiffness and/or pushability, some portion of the sensor circuit (especially but not limited to the signal return conductor) may have double functions both mechanically and electrically.

Within the context of a minimally invasive medical procedure relying on endoluminal (e.g., endovascular, or more particularly, neurovascular or cardiovascular) guidewire navigation, there may be more than one sensing modality in place. For example, external imaging device 510 is optionally provided, which is optionally an X-ray imager. Ionizing radiation-based imaging, while well-known and commonly used as a way of monitoring endovascular instrument positioning, has associated exposure risks. In some embodiments of the present disclosure, position determination using a variably resistive sensor probe 501A is used to allow reduction of ionizing radiation exposure, e.g., reduced imaging frame rates (e.g., 5 Hz instead of 30 Hz, or another factor of frame rate reduction). To maintain an acceptable level real-time feedback, interpolation of probe position through the “missing” frames optionally uses spectrally multiplexed position sensing to infer changes in position, e.g., from the previous X-ray “key frame” image. Sensor-inferred positions of the sensor-equipped guidewire are shown to a surgeon for the period between each directly imaged positions, to help maintain their positional awareness.

Each new X-ray key frame also optionally serves as a check on the accuracy and calibration of sensor data-based position calculations, optionally implemented by external processing unit 500. Optionally, adjustments are made to (for example, calibration is corrected for) the next round of position calculations which would have corrected errors noted during the previous round. Additionally or alternatively, the frame rate of ionizing radiation imaging is adjusted (e.g., via communication between external processing unit 500 and imaging device 510) according to the amount of error accumulated during the interpolation stage. If error is small, the frame rate may optionally be decreased, and if it is large, the frame rate may optionally be increased. This provides a potential advantage for maintaining a preferred tradeoff between exposure risks and reliance on more indirect methods of device tracking with the potential for gradual error accumulation. Whatever the sources of the position information at any given moment, they are optionally integrated by external processing device 500, and provided (e.g., as a live-updating display) to user interface 520. The live updating display may indicate overall context of the field of device navigation (e.g., in the form of a recently acquired X-ray image from imager 510, or another image, for example as may be obtained in the form of a CT scan or MRI image).

Additionally or alternatively, the sensor 501 itself may incorporate more than one type of sensing material. For example, it may comprise both magnetoresistive and piezoresistive material. The piezoresistive inputs potentially allow assessing sensor curvature (e.g., with more stress or strain corresponding to greater curvature), placing further constraints on overall position finding. Piezoresistive material optionally comprises a separate (e.g., parallel-wired) channel from the magnetoresistive material, and/or portions of piezoresistive material may be wired in series with magnetoresistive material.

A potential complication of endovascular navigation, particularly within increasingly fine vasculature, is the risk of perforation. Perforation in turn may become more likely as forces on the instrument being navigated (e.g., a guidewire) increase. This is another use case for piezoresistive material (which may be configured to be sensitive to such forces), optionally provided on sensor 501, in some embodiments, with or without the concomitant provision of magnetoresistive-based navigation capabilities.

It may be noted that through a bend, there are potentially both compressive and tension forces experienced simultaneously. Along a longitudinal extent of a tool, a strip of sensing material may be wound so that it alternatively extends for brief distances on all of its sides. This potentially helps to distinguish directions of flexure and twisting through different degrees of freedom (e.g., up/down, left/right, and twisted).

Potentially, wall contact information (e.g., contact with a wall of vasculature 506) is generated from environmental sensing, e.g., by noting the pattern of strain build up along a sensor. It may become clear, for example, that regions proximal to some longitudinal position of probe 501 are under stress, while regions distal to it are not. This is potentially indicative of the instrument exerting potentially damaging forces at a position away from the distal-most tip itself.

In blood vessels 506, thermoresistive sensing may be used to investigate flow conditions surrounding particular portions of the probe. For example, inducing mild resistive heating into the probe warms it up, potentially at a heating rate and/or up to a temperature which is affected by how well heat is being carried away from the probe. This may in turn be affected by a cooling effect (or lack thereof) dependent on surrounding flow. The rate of return to baseline temperature potentially is similarly affected. Flow conditions may in turn be indicative, e.g., of which side of a blood vessel 506 a probe 501A carrying the sensor 501 is pressed up to.

With the availability of a rich dataset of shape, position, and/or contact-related sensing data from all along the length of a probe, there are potential advantages for automatically assisted navigation of a probe. For example, there may in general be available a plurality of degrees of freedom used during the advance of a longitudinally extended probe 501A through a restricted lumen such as vasculature 506—forward/backward, twisting, and optionally steering with one or more degrees of freedom. In some embodiments, selection of an optimal order and/or amount of actuation of these degrees of freedom is informed by how the probe 501A itself responds to inputs, based on this sensing. For example, there may be a preference to select in each moment the most “slippery” motion—the one that best minimizes overall strains on the device while still allowing it to advance forward. In some embodiments, for example, small manual or automated trial motions are attempted, each receiving rapid feedback from along the probe's longitudinal extent. Motions actuated by the degree of freedom with the recent “best” performance may be noted and optionally recommended (e.g., on a display). In automatic embodiments, the movements may be executed, amplified, and/or repeated, and the process of testing repeated.

Another method of variable resistance-based navigation may be implemented based on thermoresistive properties. Methods exist for remotely inducing focal heating; for example, high-intensity focused ultrasound, or another form of energy which can be focused using lensing and/or phased array methods. The heating energy need not be elevated to destructive levels, however. Instead, it can be focused to create one or more mild hot spots, different enough from the surroundings to generate thermoresistive changes. The hot spots may be scanned (e.g., by operation of transducer 502A under the control of transducer controller 503A) in the region of a probe to determine its position (the probe gets hot where and at the moment that the energy focus intersects it), and/or moved along a preferred path of the probe (and slightly in advance of the probe itself) to “lead” the probe toward a preferred position. This type of “tropism” (end-seeking) navigation method may be suitable for adaptation to automated control methods, by setting up a clear navigational signal as a gradient that indicates the desired direction of movement, and provides a readily measurable parameter for evaluating progress.

It may be noted, for example, that the longitudinal extent of the sensor 501 provides a large target for the energy focus to find some portion of, and then a pathway that allows guiding movement of the focus from that portion to the distal end of the sensor. From that state, the energy focus can then be switched over to the role of leading. A correct direction and safe distance to next move the energy focus can be verified, for example, by intermittent imaging, there being periods in between image taking wherein the advancing probe is monitored through sensing as it catches up to the focus. If the probe (which can be seen by the imaging) is known to be at the focus, then the focus itself can be moved by a known amount judged from the image, and the probe again moved until it reaches the focus.

A similar type of leading navigation is potentially available with other manipulations and sensing. For example, suitably focused EM fields generated by transducer 502A (this time as a magnetic field generator) and a magnetoresistive sensing material on probe 501A may be used. A probe 501A may, for example, be navigated to seek a field region having a certain phase, direction, and/or magnitude in a gradient established by a movable electromagnetic field generated from transducer 502A, optionally without necessarily knowing exactly where the probe is at every moment, but still being certain from real time measurements that the target is being approached, without being overreached.

In some embodiments, an optional robotic driver 521 is provided, configured to manipulate probe 501A under the control of processing unit 500A. For example, probe 501A is manipulated with respect to one or more of: its distance of longitudinal advance, rotation about its longitudinal axis, and its steering articulation angle. Optionally, control over one or more additional degrees of freedom is provided, e.g., for use in tool actuation, and/or additional steering degrees of freedom.

Control, in some embodiments, is guided according to sensor data, and in particular according to the sensed shape and/or position of sensor 501. Optionally, data from additional sensing modalities (e.g., from imaging device 510) are integrated, e.g., in order to assist referencing of sensed shape and position to a current anatomical state of the region being navigated (e.g., relative position of a navigational target such as a branch of a bifurcation). In some embodiments, robotic control is provided in the form of navigational assistance for certain tasks. For example, robotic control may be activated for assistance in steering tasks and/or passing obstacles, with navigational guidance overall being apportioned to manual operations.

In some embodiments, robotic control is used to help in manual selection of movements. Optionally, manual control of probe 501A is performed through controlling actuators of robotic driver 521 itself. Optionally or additionally, robotic driver 521 may be configured to receive indications of forces exerted manually on probe 501A from outside of its control, e.g., it may sense changes in advance or rotation, and or sense tension and/or compression inn probe 501.

In some embodiments, the assistance is in the form of governing the dynamic characteristics of the device in response to inputs; e.g., reducing sensitivity to steering inputs when it is judged (automatically, and/or in response to a user input) that the operator is and/or is expected to be searching for a steering angle within a small range. Automatic judgements may depend, for example, on the recent history of user adjustments (e.g., an oscillating input may indicate difficulty in finding a needed steering setting). Additionally or alternatively, the judgement may depend on a recent history of sensor shapes and/or positions, where again, oscillation may indicate a potential difficulty. In some embodiments, sensor-observed mismatch between attempted inputs and observed positions of sensor 501 is used to help identify situations in which assistance may be useful.

In some embodiments, the assistance comprises executing a plurality of micro-movements which help the robotic system to assess the current state of the device, and which inputs may assist further progress toward a goal such as a target position. Effects of the micro-movements are optionally measured based on assessment of the shape and/or position of sensor 501. For example, a distal tip of sensor 501 may move or not move; if it moves, it may move in an intended direction, or not. Changes to the shape of sensor 501 may indicate how manipulation forces are being absorbed. For example, upon receiving an advancing input, increased bending of sensor 501 up to but not past a certain location may indicate that there resistance to sliding motion concentrated on that location. Such findings may be indicated to the operator, e.g., via user interface 520.

In another example, changes to the shape of sensor 501 in response to micro-movements may help determine combinations of inputs which may be helpful. For example, deflections in the shape of sensor 501 along its length may change slightly as small movements (e.g., advancing/retracting and/or twisting) are made which adjust forces and/or points of contact. Adjustments which tend to move the sensor 501 toward a more favorable starting point for receiving further inputs like steering angulation are optionally detected. The operator may choose, for example, to carry out those adjustments themselves (upon receiving suitable indications, e.g., via user interface 520), or to have the robotic driver 521 itself amplify promising adjustments into larger movements.

### Sensor Designs

### FPC Sensor Design

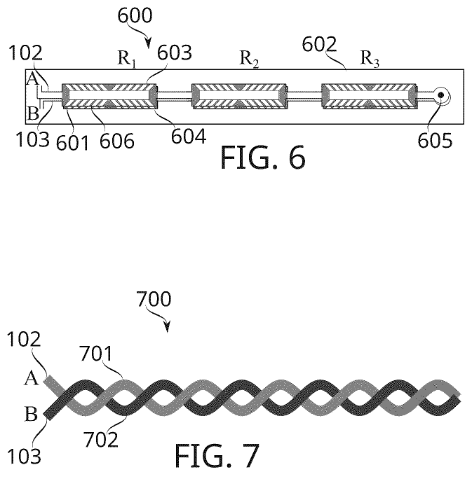

Reference is now made to FIG. 6, which schematically represents a two layer sensor FPC with discrete “barber pole” resistive elements 603, according to some embodiments of the present disclosure. In some embodiments, the “barber pole” arrangement comprises a plurality of substantially parallel diagonal stripes of magnetoresistive material 601, joined through inter-spaced conductors 606 connecting one stripe to another at an oblique angle (e.g., 45 degrees) relative to the longitudinal axis of the FPC. This configuration forces an obliquely oriented electrical current between the resistive stripes. This potentially exploits anisotropic properties of a magnetoresistive film to promote sensing of fields with orientations away from, e.g., a longitudinal axis of the probe.

In some embodiments, a sensor 600 comprises sensing material 601 arranged in discrete units 603, optionally without any introducing discrete reactive components in between. In the example shown, sensing material 601 is encapsulated by a flexible printed circuit (FPC) ribbon 602.

A potential advantage of breaking the sensing material 601 (e.g., material in the form of a film) into discrete units is focusing resistances into more distinctly localized locations along the longitudinal extent of the sensor 600.

This potentially makes sensor impedances easier to determine from measurement data; e.g., the approximation that they can treated as N discrete resistances Rᵢ more closely reflects the physical arrangement of the device. This may make the resistances Rᵢ easier to determine, compared to the case of a continuous and homogeneously distributed resistance and/or resistance function R(x).

In some embodiments, sensor 600 comprises a two layer FPC circuit, having a plurality (e.g., N=3 as shown, but optionally more, for example, 10, 20, 30 or more) “barber pole” discrete units 603. Discrete units 603 are provided as traces comprising sensing material 601, optionally in the form of a resistive film (shown as the top gray layer).

The second (bottom) layer, in some embodiments, contains a signal return path 604. This can be made of more units 603 (e.g., as shown) or of an ordinary conductor such as copper, and/or of a sensing material arranged in another fashion. That may be of the same, or optionally of a different sensing material, although in the case of a different sensing material, it may be treated as an additional resistance, instead of as one of the resistances Rᵢ shown.

Connections are soldered or otherwise joined to the FPC at terminals 102, 103 (i.e., pads A, B). The layers may be connected at the distal end of the FPC using a via 605. A termination resistor Rₑ or other termination element (not shown) may be added to the circuit just before the Via.

In some embodiments, FPC sensor 600 contains a special layer which may be made of special resistive material (for example, a magnetoresistive material) instead of plain copper. This material can be incorporated in the FPC manufacturing process similarly to the inclusion of a standard copper layer, and may undergo the same manufacturing processes as copper (such as etching). This allows the resistive material to be shaped and laid out precisely along FPC ribbon 602.

In some embodiments, sensor FPC is wound helically around a cylindrically structured device (e.g., a pipe-structured device such as a catheter). The configuration can be made such that each consecutive resistive element (which amounts to a sample point in space of the magnetic field) sits on a different side of the pipe, such that the elements units are oriented in at least two different orthogonal directions along the pipe. Since, in embodiments using EM sensing, a magnetoresistive FPC sensor may have anisotropic sensitivity to field direction, this potentially makes the magnetic measurements more informative, assisting determination of the sensor's position over its full longitudinal extent. In such configuration, it might be important to know the exact geometry of the sensor winding around the pipe. This geometry can be determined in supervised or unsupervised calibration processes, by measurement, by manufacturing process design, or another method.

Insofar as measurements may be invariant to rotation of the FPC about the FPC's length axis (although, as described hereinabove, they may actually be sensitive to this, depending on device configuration), it may not be practical to solve the twist of the curve in space using them, although this information may be useful for some applications.

In some embodiments, accordingly, an additional resistive film layer may be added to the FPC, optionally identical to the first layer but with an offset, e.g., to an opposite side of the sensor. Sensor measurements are made from each layer, and positions computed for them both. When the FPC is twisted, the twist is potentially noticeable as a discrepancy between the two solved curves. The final catheter position (and twist) can be solved by combining the two solve position curves and adding the difference vector between them, which determines the positional twist of the curve. Another method of detecting device twist is described, for example, in relation to FIG. 4, herein.

### Twisted Pair Sensor

Reference is now made to FIG. 7, which schematically illustrates a twisted-pair variable resistive sensor configuration, according to some embodiments of the present disclosure.

In some embodiments, a sensor 700 comprises electrically isolated variably resistive wire 701 may be used which is made of material similar to that of the variably resistive film (for example, enameled wire). The variably resistive wire 701 may be twisted to create a twisted pair between the wire 701 and another wire 702. Wire 702 is optionally itself variably resistive, or made of a plain conductor.

This creates a circuit similar to that of, for example, FIGS. 1 and 3. The two wire portions 701, 702 are inductively and capacitively coupled and optionally terminated with a Rₑ resistor or other terminating element. The twisted pair characteristic impedance can be calculated using known formulas, and the continuous resistance R(x) can be computed from total impedance measurements Zⱼ using the methods described above. The construction of such sensor is relatively simple and its footprint is potentially ultra-small (for example, smaller than 0.5 mm, smaller than 360 μm or about 0.014 inches, smaller than 250 μm, or another diameter). The number of twists in the self-twisted pair affects the characteristic inductance of the circuit and can be changed according to the application. It (twisting) may be non-uniform along the length of sensor 700. In the extreme case, the wire may not be twisted at all, in which case the two wire portions 701, 702 may extend one next to the other in parallel.

In some embodiments, reactance (e.g. inductance and/or capacitance) differences arising from varied twist density as function of position along the sensor are used to help electrically differentiate different regions, so that alterations in resistance along the sensor are distinguishable, e.g., as described in more generally in relation to FIG. 3 with respect to the variation of intrinsic reactances along the sensor length. In some embodiments, conductive material used in one or both strands of the twisted pair is selected for its mechanical properties, e.g., its steerability, torqueability and/or pushability. More magnetoresistive and more mechanically stiff portions of the twisted pair may alternate, optionally with the other member of the pair being constructed to provide the complementary set of characteristics along same respective extents of the sensor. In some embodiments, the material of at least portions of one or both of the twisted pair members is selected for its properties as a magnetic core, e.g., a high-permeability non-linear material; made for example, of permalloy, supermalloy, mu-metal, cobalt alloy, or any other high permeability non-linear magnetic core. This is optionally arranged to enhance changes in circuit resonance characteristics as a function of magnetic field strength.

Twisting by the members of the twisted pair is not necessarily symmetrical. For example, one strand may be thick enough that the other strand winds around it, while the thicker strand deflects by smaller amounts per twist, or not at all. The asymmetry may approach or actually provide the geometry of an inductor coil having a straight internal core, and a wound coil around it. The magnetoresistive material is optionally provided as part of the more coil-like portion, and/or as part of the more core-like portion. In some embodiments, which strand of a pair is thicker and which is thinner is the same all the way along the probe. In some embodiments, the strands alternate construction along their lengths.

Optionally, the more core-like portion is twisted with twists that go around itself, rather than around its partner lead. For example, a film-like magnetoresistive material itself may be constructed in a fashion (e.g., as a layered film, with limited ductility, and/or having a width notably larger than its thickness) which is difficult to wind as a coil layer with a pitch small enough to make appreciable inductance contributions. As may be appropriate to keep its maximum cross-sectional dimension small, the material may optionally be twisted with a relative long pitch, optionally surrounding a supporting material. The supporting material's properties as a magnetic core may be high-permeability and non-linear, or otherwise.

Material provided for a thinner strand of an asymmetrically constructed twisted pair may be suitable to be wound into a fine-pitch coil (e.g., as thin copper or gold wire, e.g., in the range of 40-54 AWG). Optionally, it is wound along the sensor in a pitch that is different in different sections (continuously or variably), and/or in different numbers of layers in different sections. This provides reactance variation which may be used to help distinguish resistivity changes in different magnetoresistive sections along the probe.

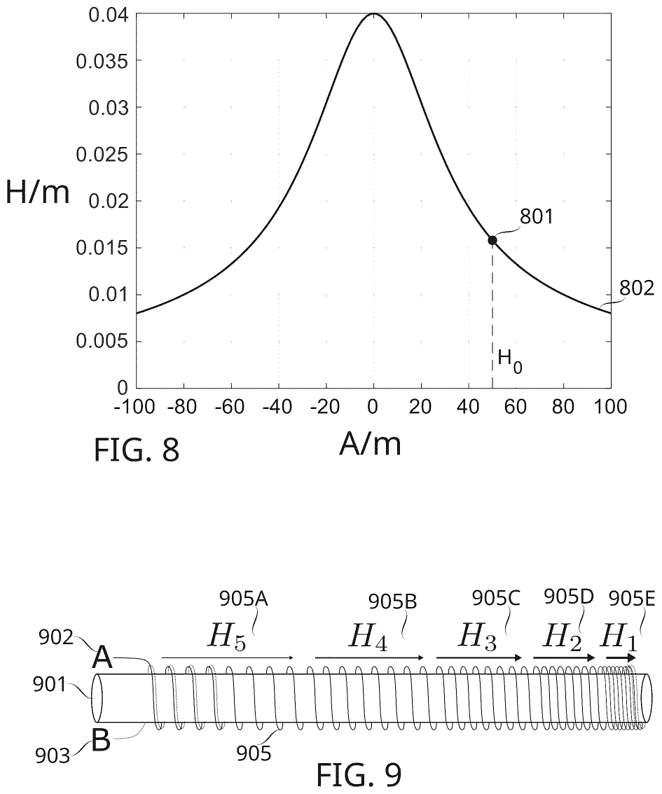

Methods of spectral decomposition analysis described with respect to magnetoresistive sensing are based, in some embodiments, on how local circuit resonance properties change along a sensor's length according to interactions with a magnetic field. In magnetoresistive sensing, these resonance property changes are the result of resistance changes, with the spectral decomposition being supported by an overall inhomogeneity in baseline resonance properties along the sensor's length, e.g., as determined by resistance, inductance, and/or capacitance. In some embodiments, magnetoresistance changes are supplemented, or optionally replaced, by magnetic field interactions with another electrical parameter of the sensor. For example, the use of magnetoinductive properties as the basis of magnetic field sensing are described with relation to the embodiments of FIGS. 8-14. In relation to those figures, a method of analyzing measurements is described which uses currents to generate location-dependent offsets (e.g., from a zero-magnetizing field baseline condition) of the permeability of high-permeability non-linear core materials. However, it should be understood that there is also an option, used in some embodiments, to apply the spectral decomposition method described for magnetoresistive sensor probe embodiments using magnetoinductive sensor probe embodiments additionally and/or instead. For example, the various inductances L₁ . . . . Lₙ of FIG. 1 and/or ΔL of FIG. 3 may include a magnetoresistive contribution (e.g., from an inductor with a highly permeable, non-linear core) that affects circuit resonance locally. Optionally, constructions described, e.g., in relation to the magnetoinductive proves FIGS. 9-11 and/or 13 are used in such embodiments.

As mentioned above, certain physical quantities affect the resistance of different type of films. However, sometimes a plurality of physical phenomena may affect the film, which is undesired if only a specific physical quantity is to be measured. For example, a magnetoresistive film may also be affected by temperature and strain which may affect the magnetic measurements as parasitic effects. In order to compensate for these effects, a symmetric (but non-magnetoresistive) layer of the same geometry as the magnetoresistive film may be added to an FPC sensor and its resistance can be measured at multiple frequencies using the same methods described above. Since the layer is not magnetoresistive it will only measure the parasitic effects: for example, thermo-resistive and strain. These measurements (suitably calibrated) can then be subtracted from the measurements of the magnetoresistive layer, thus compensating for the undesired parasitic effects and improving the magnetic measurements.

In a magnetoresistive sensor, the magnetoresistive film may be magnetized in order to be able to sense the magnetoresistive effect. To accomplish this, an external processing unit 100, 500 may send a high electrical current pulse through the curve sensor's ports and into the magnetoresistive films to magnetize the films. The pulse can be long enough (for example 100 μs long) such that it is almost considered as a DC current with which the reactive elements of the circuit need not interfere.

Reference is now made to FIG. 8, which plots a magnetization (or magnetic strength, in A/m) to permeability (or “inductance per meter” in H/m) relationship of an example magneto-inductive coil, wrapped around a high permeability non-linear magnetic core, according to some embodiments of the present disclosure.

In a magneto-inductive sensor according to some embodiments of the present disclosure, a high permeability non-linear magnetic core with an induction coil wrapped around it interacts with the magnetic field at points along the (typically curving) core's longitudinal axis. Current in the coil experiences inductance which changes according to the strength of the total magnetic field which the non-linear magnetic core material experiences. The actual “experienced strength” may be judged by the resulting effects on current, which potentially vary from effects expected from the magnetic field vector as such, due to factors such as anisotropic sensitivity of the core material. The change in inductance is due in turn to the dependence of a coil inductor on the magnetic field permeability of its core material.

Accordingly, suitable measurement of how current flows through the sensor provides an indication of the magnetic field's properties. In particular, by utilizing a pre-calibrated permeability to magnetic field relation, the magnetic field can be computed.

In order to get the maximum sensitivity from the magneto-inductive sensor, it may be measured around the linear regime of the inductance curve 802—that is, it is measured in a region where the change in permeability is relatively linear as a function of changing electromagnetic field strength (and more particularly, magnetization field strength). As shown in FIG. 8, this relatively linear regime is displaced to either side of zero field magnetization field strength. However, driving a current through the coil applies a bias magnetization H₀ (marked at graph location 801), which is preferably selected to be near the middle linear regime of the curve 802.

The sensor's inductance can then be measured, with shifts from the bias location 802 resulting in a change in permeability (vertical axis of FIG. 8) which can be referenced to the amount of added or decreased magnetic field strength in the environment (displacement from location 801 along the horizontal axis of FIG. 8). As implemented for some embodiments of the present disclosure, the relationship may be described in another way, for example, as being among two different but related parameters. For example, permeability in H/m may be replaced by reference to inductance per coil, or linear inductance density (also in H/m) as such. Magnetization in A/m may be referenced to current (at least when current induces it), or to another parameterization of the electromagnetic field such as the magnetic field vector.

The sensor's inductance is optionally measured using RLC oscillation methods, digital oscillation methods or any other suitable method. For example, in a digital oscillation inductance measurement method, a bias resistor R_{b} determines the electrical bias current (and therefore also the bias magnetization of the coil). The bias introduced by the inductance measurement itself may be determined by comparing to measurements in which the sensor is reverse biased and sensed around negative bias magnetization −H₀.

In some embodiments of the present disclosure, a flexible magnetic core is used so that the magneto-inductive sensor built around it can be flexible. For example, the magnetic core is constructed as a flexible thin wire (for example, 0.2 mm in diameter). The flexible core wire is made of a high permeability flexible material such as supermalloy. One example of a supermalloy is composed of nickel (75%), iron (20%), and molybdenum (5%). The one or more coils of the sensor are wrapped around the core wire. The conductor of the coil(s) comprises, for example, 38 AWG enameled copper or gold wire, or thinner (e.g., 40 AWG-54 AWG).

In the setting of a single long coil, as discussed above, the total inductance of the sensor can be measured and the total magnetic field along the sensor's flexible curve can be sensed. This readily provides a measurement of the average (e.g., algebraic average) magnetic field experienced by the whole sensor (though the average is potentially weighted somewhat due to non-linearities present in the system).

In some embodiments of the present disclosure, additional features are provided to enable distinguishing of the potentially different magnetic field values which exist along the sensor's curve. The magnetic core's non-linearity is exploited in order to decompose the sensor's inductance measurements into different inductances along respectively different portions of its length.

Reference is now made to FIG. 9, which schematically represents an inductive sensor comprising 5 discrete coils connected in series, according to some embodiments of the present disclosure. Due to the coils' different pitches, each coil exerts a different magnetization force on the segment of magnetic core 901 which it surrounds.

The 5 discretely labeled inductive elements 905A-905E of FIG. 9 (labeled also with their associated magnetization forces H₁ to H₅) comprise coils wrapped around a high permeability non-linear magnetic core 901, in increasing winding pitches, and connected in series. In the example as depicted, the coils are wrapped with wire 905 from left to right, and then wrapped in a second layer from right to left (indicated by the dotted lines) so that the two sensor's coil terminals 902, 903 (also marked A, B) are both located on the same side (this for example can be the device's proximal end).

In a general for such a sensor, each of the N discrete coils exerts a magnetization force Hᵢ(I)=kᵢI on the surrounded magnetic core segment, which depends on the electrical current I flowing through the coil (which is identical for all coils). kᵢ is a constant factor which depends on the geometry of the coil, and in particular is in inverse proportion to the winding pitch of the coil (the denser the coil, the smaller the pitch, and the higher kᵢ). Denoted by Bᵢ is the component of the external magnetic field at the position of the i^{th} coil oriented in the direction of coil winding. The total magnetization force which is exerted on the i^{th} core segment is therefore:

$${H}_{i}^{tot} ( I , {B}_{i} ) = {H}_{i} ( I ) + {B}_{i} = {k}_{i} I + {B}_{i}$$

Accordingly, the total magnetization force Hᵢ^{tot} depends both on the electrical bias current I and external magnetic field Bᵢ. The inductance of the i-th magnetic coil can be computed using the magnetization-to-inductance relationship, given by the pre-calibrated curve μ(H) (of which an example is shown in FIG. 8):

$${μ}_{i} ( I , {B}_{i} ) = {l}_{i} \cdot μ ( {k}_{i} I + {B}_{i} )$$

Where lᵢ is the length of the i-th coil element (which is known in advance or found in pre-calibration). Finally, the total measured inductance of the complete coil (comprising individual coil elements, connected in series), for a given bias current I and given external magnetic fields Bᵢ is:

$${μ}_{tot} ( I ) = {μ}_{tot} ( I , {B}_{1} , {B}_{2} , … , {B}_{N} ) = {\sum }_{i = 1}^{N} ( {l}_{i} \cdot μ ( {k}_{i} I + {B}_{i} ) )$$

Over a given short period in time (e.g., a few hundred microseconds, for example, 100-1000 usec), {Bᵢ} can be treated as constant for purposes of these measurements; at least, so long as the transmitted magnetic fields are relatively low in frequency, e.g., <200 Hz. The various values of {Bᵢ} are unknown and need to be solved from the integrated inductance sensor data. They cannot be solved from a single measurement and μₜₒₜ's equation alone. However, I is completely controlled by the sensor, so that the sensor can measure the total inductance μₜₒₜ(I) of the complete coil under M different values of I. For example, 16 different current values are used in measurements, distributed (e.g., uniformly distributed) between −100 mA and 100 mA. In some embodiments, within the same range or a different range of currents, at least 8 current values are used, at least 12, at least 16, at least 20, or another number.

The measurements with different currents yield a system of M non-linear equations, due to the non-linearity of μₜₒₜ(I):

$${\sum }_{i = 1}^{N} ( {l}_{i} \cdot μ ( {k}_{i} {I}_{1} + {B}_{i} ) ) = {μ}_{tot} ( {I}_{1} ) \\ {\sum }_{i = 1}^{N} ( {l}_{i} \cdot μ ( {k}_{i} {I}_{2} + {B}_{i} ) ) = {μ}_{tot} ( {I}_{2} ) \\ ⋮ \\ {\sum }_{i = 1}^{N} ( {l}_{i} \cdot μ ( {k}_{i} {I}_{M} + {B}_{i} ) ) = {μ}_{tot} ( {I}_{M} )$$

This system can be solved, for example, using non-linear optimization approaches (e.g., Gradient-Descent, Levenberg-Marquardt). To ensure that the system is solvable and has a unique solution, {lᵢ}, {kᵢ} are preferably chosen to maximize differences between the equations. For example, in the degenerate case where all {lᵢ} and {kᵢ} are equal, it is clear that the system is unsolvable due to the symmetry of {Bᵢ}, as they can be swapped without affecting the total measured inductances.

Adjusting {lᵢ}, {kᵢ} appropriately helps ensure the system is solvable. This is achieved, for example, by wrapping the coils with sufficiently varying pitches, e.g., as depicted in FIG. 9. For example, the coils can be wrapped with an exponentially increasing pitch. For example, in the case of 5 discrete coils, the pitches may be: 0.01 mm, 0.02 mm, 0.04 mm, 0.08 mm, and 0.16 mm. Alternatively, all the coil elements may be wrapped with minimal pitch (dense wrapping), but each coil can contain a different number of layers. A conductive wire (for example, 44 AWG enameled gold wire) can be wrapped from left to right, creating the first layer of the coil elements, then wrapped back from right to left. It can then be wrapped again from left to right, stopping before the most distal coil, then back to left. It can then be wrapped again from left to right, stopping before the 2^{nd} most distal coil, then back to left and so on. In such manner the coils will contain 2, 4, 6, 8, 10 . . . layers which will be reflected as kᵢ=2k₀, 4k₀, 6k₀, 8k₀, 10k₀, . . . in the magnetization forces Hᵢ.

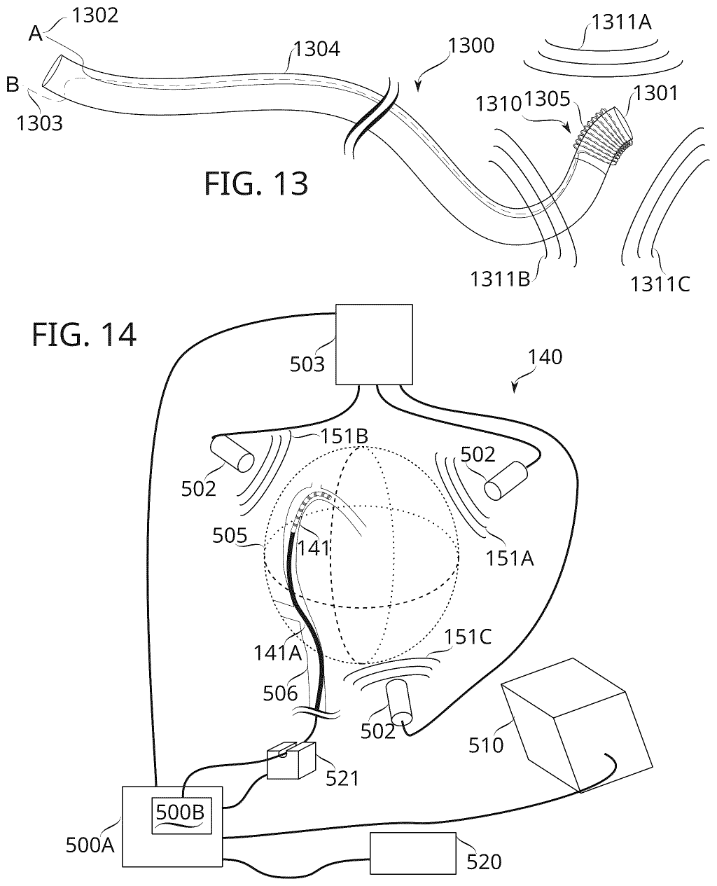

Full 3-D spatial localization (optionally with additional degrees of freedom specifying local orientation, too) may be provided when each location in space can be identified by a unique electromagnetic signature, or at least, electromagnetic signatures which are distributed so as to allow only one plausible compatible configuration (location and shape) of the curve sensor. With a single magnetic field (which can be variable or constant, e.g., as produced by a DC current), this is generally impractical, unless the shape and position of the curve sensor are known to be confined in some other way, e.g., confined to a single plane. In that case, the fixed length of the probe may effectively constrain what solutions can account for variations along its length in the single magnetic field's strength. Constraints of this and other types can also be used to assist 3-D spatial localization, as described in relation to FIG. 13.

For more general case, a plurality of magnetic fields may be provided, for example as described in relation to FIG. 13. Magnetic fields crossing through a region but oriented in different directions can “tag” it with a plurality of coordinate values. For example, three magnetic fields which are roughly mutually orthogonal in a region can set up coordinates arranged like the coordinate axes of a 3-D Cartesian space. The magnetic fields are not necessarily orthogonal, however, nor do they necessarily even vary along straight lines. More fields than three may be provided, e.g., to help ensure that positions in all regions of interest can be sufficiently identified and distinguished from one another, e.g., distinguished with a resolution suited to navigation needs. Strengths of the magnetic fields may be known, for example, from a model modelling the magnetic fields set up by a particular operating configuration of transducers. The model may be generated by any suitable combination of measurements, calibration assumptions, and/or theoretical calculations, as is known in the art.

A flexible approach to combining the magnetic fields in a single location while allowing them to be distinguishable varies each field at a different frequency. Spectral decomposition can then isolate their various influence. The frequencies used can be fairly low, e.g., less than 500 Hz. Additionally or alternatively, the magnetic fields can be combined by temporal multiplexing, e.g., by alternating their activation fully between on and off.

Reference is now made to FIG. 10, which schematically represents a flexible sensor comprising a single coil made of 8 discrete coil elements connected in series, according to some embodiments of the present disclosure.

In this case, each coil element 1005A-1005H (associated with a magnetization force H₁-H₈) is magnetized by a magnetization force Hᵢ which is determined by the external magnetic field Bᵢ and the coil configuration which affects kᵢ and lᵢ (number of windings, winding pitch, number of layers etc.).

To construct such a configuration, wire 905 may be wrapped in sections corresponding to each coil, and the sections joined after winding. Inter-coil connections are not shown. To facilitate joining the coils, there may be a pair of independent windings in each of coil elements H₁-H₈, with one lead on each side of each coil leading distally from lead end 1002 (also labeled A), and a second lead on each side of each coil leading proximally toward lead end 1003 (also labeled B). Optionally, each coil has two ends, with wire 905 extending straight, e.g., from lead end 1003 to the distal (right-side) end of core 901 to attach to the distal end of the last winding (H₁) there.

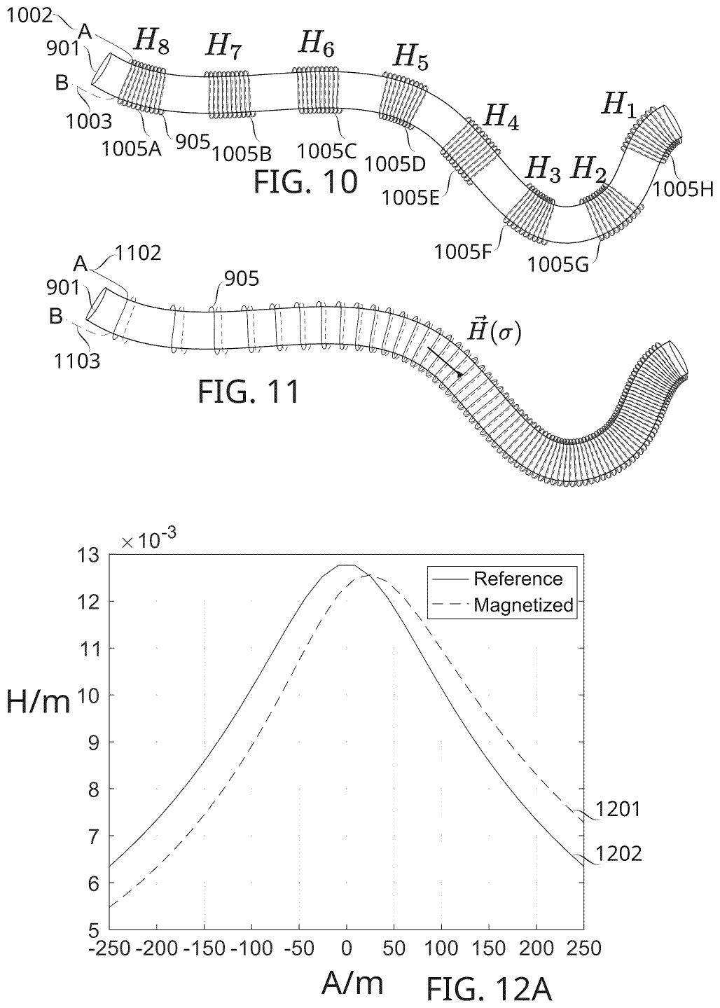

Reference is now made to FIG. 11, which schematically represents a flexible sensor comprising a coil with continuously decreasing winding pitch, according to some embodiments of the present disclosure.

In the example in FIG. 11, the winding pitch decreases monotonically (the winding density increases accordingly) until the coil reaches the distal part of the sensor. The rate of pitch change itself can be constant or varying. Winding of wire 905 may be performed, for example, from lead end 1102 (labeled A) to the distal end with decreasing pitch, and then back again to lead end 1103 (labeled B) with increasing pitch. Alternatively, the pitch may, for example, increase towards the sensor's tip, or the pitch may vary non-monotonically, or non-continuously or in any other similar way.

Lacking well-defined inflections in winding or spacing to mark distinct units (as, e.g., shown in FIGS. 9-10), the magnetization force per unit length in this case does not change step-wise. To analyze this, the magnetization force applied on the flexible core at each length parameter σ∈[0,L] along its curve (where L is the complete coil's total length) can be given by a continuous function H(σ). Similarly, the winding pitch can be given by P(σ) (where σ is same as before).

The continuous winding pitch P(σ) can optionally be modeled as a spline interpolation between a discrete set Pᵢ, corresponding to length parameters σᵢ∈[0,L]. The number of points in the discrete set can be selected according to what is needed to fo!r!0 coil; e.g., more points used for less-smooth or more direction-changing pitch variation patterns.

Assuming changes in the external magnetic field along the sensor's curve B(σ) are also well represented by a spline curve set of order N (usually the case), both the magnetization force and the external magnetic field can also be modeled as a spline interpolation between discrete points, Hᵢ, Bᵢ at σᵢ. If necessarily, the size of the set of spline points can be increased according to requirements imposed by irregularities of the magnetic field variations.

With this transformation adopted, the problem of measuring the continuous external magnetic field along the sensor curve of the continuous sensor coil in FIG. 11 reduces to the problem of finding a set of discrete magnetic fields Bᵢ at points σᵢ along the sensor's curve, which can be solved, e.g., as explained for in relation to FIGS. 9-10.

It should be understood that there are other ways of providing inductance which is variable along a longitudinally extended axis of a probe, alternatively or additionally to the use of one or more of those described in relation to FIGS. 9-11. Apart from variations in coil winding and/or coil composition, the core can be varied in composition and/or dimensions. For example, in some embodiments, the core diameter varies along the sensor's length. In some embodiments, the magnetic core's alloy composition varies along the sensor's length.

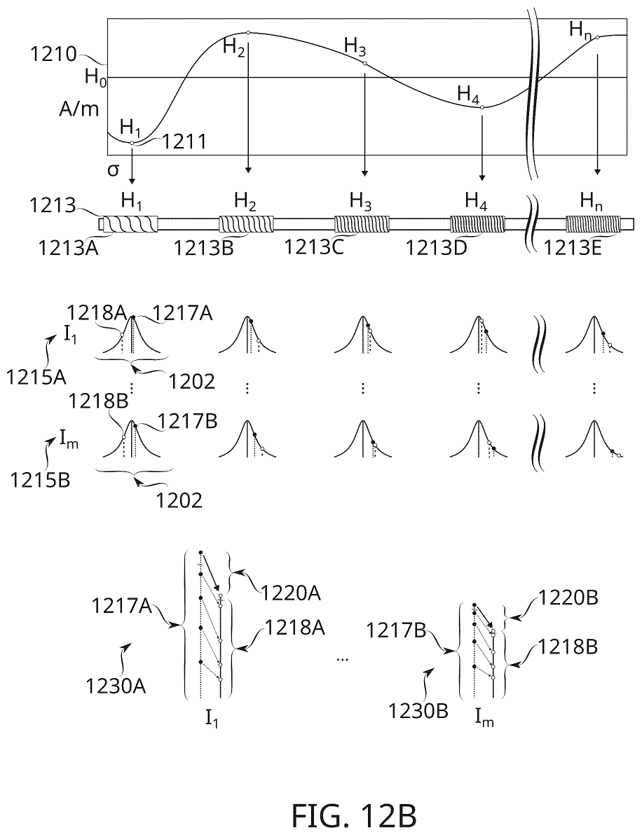

Reference is now made to FIG. 12A, which plots a magnetization to inductance relationship of an example magneto-inductive coil with varying pitch, under an externally applied magnetic field which varies along the sensor's curve, according to some embodiments of the present disclosure. Reference is also made to FIG. 12B, which schematically indicates features of data acquired using a magneto-inductive sensor 1213, according to some embodiments of the present disclosure.

In the example of FIG. 12A magnetization (the horizontal axis in A/m) is related to permeability (“inductance per meter”, vertical axis in H/m) relationship calculated for a flexible curve inductive sensor made of 4 discrete sub-coils connected in series, wrapped around a high permeability non-linear flexible magnetic core, each discrete coil having a different winding pitch.

The coils can be formed, for example, by winding a single long conductive wire with varying winding pitches in 4 discrete steps. A measurement circuit electronically samples current and/or voltage through the coils assembly using just 2 terminals which are the endpoints of the long wrapped wire forming the coils.

Curve 1202 shows the reference condition for the probe without an externally applied magnetic field. The probe bias may be set to any suitable part of this curve, for example, 150 A/m.

The example sensor is then positioned inside a spatially varying magnetic field such that each of the discrete coils “sees” a different magnetic field.

In the example of FIG. 12B, graph 1210 shows various magnetic fields H₁ through Hₙ, each corresponding in its position to one of coil regions 1213A-1213E. of magneto-inductive sensor 1213. Position in graph 1210 is shown along parametric axis σ, which is parameterized by distance along magneto-inductive sensor 1213. Each offset along σ also corresponds to a position in space, e.g., a position characterized by positions r(σ)=(x(σ),y(σ),z(σ)) along the transmitter's orthogonal coordinate axes, or otherwise characterized.

The target intermediate result is measurement of the plurality of different magnetic field strengths along the length of the probe (in the case of FIG. 12A, N=4 fields; in the case of FIG. 12B, there are n fields H₁ . . . . Hₙ indicated in graph 1210).

This intermediate result can then be referred to known or predicted magnetic field strengths at different locations in space in order to determine where the probe is, and/or what its present shape is.

With reference to FIG. 12A: to obtain data for calculating the target intermediate result, measurements of inductance μₜₒₜ(I) of the complete coil under M different values of I are obtained. Curve 1201 shows a different relationship between inductance and magnetization which is calculated to be consistent with what has actually been measured. The specifics of the changes from the position of curve 1202 are due to the combined effects on total inductance due to the specific values of the external magnetic field experienced individually by the four coils.

In FIG. 12B, elements of this process are further broken down as follows. Throughout a measurement period, the field strengths H₁ . . . . Hₙ impinge upon coil regions 1213A-1213E, respectively, as noted by arrows leading between them. Rows 1215A, 1215B represent a model context for measurements of inductance per meter made at each current in a range of currents I₁ . . . . Iₘ.

Each instance of the bell-shaped curve 1202 repeated in each row represents a same inductance curve with horizontal units of magnetic field strength A/m, for example as shown in FIG. 12A.

The positions of each of the black dots 1217A relative to its respective inductance curve 1202 represent (along the X-axis) magnetization away from the center peak induced by current I₁. Along the Y-axis is shown the expected corresponding inductance per meter for current I₁ in the absence of an external magnetic field e.g., if external magnetic field strength fell along the line marked H₀ in graph 1210, instead of the curve marked H₁ . . . . Hₙ. The X-axis offset is different for each column position, since the different constructions of impedance regions 1213A-1213E result in different partial impedances for the same current. As a result, there is also a different Y-axis position for each black dot 1217A.

The black dots 1217B represent the same, but for a different current Iₘ. Generally, if Iₘ>I₁, then the X-axis offset of each black dot 1217B from the peak of graph 1202 centered on 0 A/m is also larger than the corresponding black dot 1217A in the same column. Ratios of offsets along the X-axis at different currents are not expected to be in constant proportion to the current ratio, since the current-induced magnetizing fields are themselves susceptible to permeability non-linearities.

Under conditions where the magnetic field strengths H₁ . . . . Hₙ are imposed, inductances for each current I₁ . . . . Iₘ at each position 1213A-1213E shift once again to reflect the combined magnetic field vector. In FIG. 12B, this shift is represented by hollow dots 1218A, 1218B. Along the X-axis, the shift is expected to be about the same for any given position 1213A-1213E under all measurement currents used, since the external magnetic field is not itself dependent on local permeability. However, the magnitude of shift in local permeability of the sensor along the Y-axis is different for different currents, since the starting offset is different, and different regions of the permeability curve have different slopes.

Although represented with separate values in each of rows 1215A, 1215B, the permeabilities (μ in H/m) of individual regions 1213A-1213E are not separately available in raw measurements. Instead, raw measurements sum all the inductances they contribute to for each measurement current I. The summed values correspond, e.g., to the vertical stack of inductance values corresponding to hollow dots 1218A (bottom left panel 1230A, for current I₁), and hollow dots 1218B (bottom right panel 1230B, for current Iₘ). Summed value is represented as height in these stacks. In panel 1230A, for example, the height of the stack of values 1217A represents Σᵢ₌₁^{N}μᵢ(I₁,H₀), and the height of the stack of values 1218A represents Σᵢ₌₁^{N}μᵢ(I₁,Hᵢ). The sum is optionally weighted by element length (in effect converting permeability to inductance proper) if lengths of elements 1213A-1213E are not all the same.

The differences 1220A, 1220B between stack heights for a particular current represent a total external magnetic field strength effect on inductance compared to baseline. These stack heights correspond also to points on curve 1201 of FIG. 12A—they are the “shifted” inductances.

To extract individual permeabilities (inductance if considered as length-weighted) from these measurements, the various summed heights are mathematically “explained” through the model's inductance curve, in a way that consistently proposes the same underlying values of H₁ . . . . Hₙ no matter what current I₁ . . . . Iₘ was used to produce the measurement.

As described, e.g., in relation to FIG. 9, the various inductive elements 1213A-1213E are preferably configured so that measured total inductances through a range of currents I₁ . . . . Iₘ can only be “explained” in the context of the other known parameters of the situation for particular values of externally applied magnetic field strengths H₁ . . . . Hₙ along the sensor's curve. The solution may be unique, so long as the design of the probe and measuring conditions was selected to avoid mathematical degeneracy in the solution process.

Next, the curve 1202 (that is, a pre-calibrated inductance to magnetization curve of the sensor) can be used to search for the magnetic field values which yield the measured inductance values. In this way, the plurality of magnetic field values along the sensor can be solved (in this example, N=4 values).

To proceed from this stage to the calculation of sensor position and/or curve, knowledge of how external magnetic fields area actually distributed in space is used. In some embodiments, shape and/or position sensing also comprises determining magnetic fields for a plurality of separately generated magnetic fields, so that locations in space can be individually identified according to the strengths and/or directions of the different magnetic fields which overlap within them. In some embodiments, one or more additional constraints are used: for example, the fields are constrained to be smoothly varying, and/or the shape and/or positions are constrained to be physically plausible shapes and/or positions of the probe.

### Endoluminal Device Integration

Reference is now made to FIG. 13, which schematically represents a guidewire 1300 having an integrated flexible sensor 1310 comprising a coil 1305 wound around a core 1301 which is constructed as a distal extension of the main guidewire body 1304, according to some embodiments of the present disclosure.

In some embodiments of the present disclosure, the physical principles described in relation, e.g., to FIGS. 9-11 for a flexible curve inductive sensor are used to provide a body-integrated EM sensor 1310 to a device 1300. Sensor 1310 may be provided in such a way as to replace a portion of the body of the device 1300, e.g., to replace a section of the length of the device 1300. In some embodiments, the device 1300 is a guidewire; optionally, the device 1300 is another longitudinally extended device incorporating a wire portion, for example, a catheter comprising at least one section of helically wound wire wall.

Additionally or alternatively, replacement of a portion of the host device body with the body of a sensor probe is optionally applied to a probe providing full curve sensing. In some embodiments of the present disclosure a shape sensed probe (elongated device with variable inductance density along its length) is constructed which is fully curve-tracked in 3-D in real-time relative to an EM transmitter (e.g., as further described hereinbelow, first with respect to device 1300 and then generalized to full curve sensing). The shape sensor may constitute a 40-44 AWG or thinner (e.g., 44-54 AWG) copper or gold wire wrapped around a high permeability non-linear flexible magnetic core, such as a supermalloy wire (for example, a 0.1 mm-0.3 mm core wire). The coil may be wrapped in varying pitch, and/or the core diameter may be varying along the sensor's curve to provide informatively sensed inductance curves which are indicative of the sensor's full shape in space relative to the transmitter. The sensor's coil is optionally used together with the core to provide (and potentially improve) mechanical properties associated with the probe portion of the host device, such as: steerability, torqueability, and/or pushability of the probe. The sensor's full length may be tracked (for example, 40 mm-200 mm of its distal part) or the sensor's distal end, or just the sensor's tip (in which case, it may reduce to an embodiment similar to that of FIG. 13).

### 3-D Spatial Localization of Shape and Position

### Basic Multi-Field Spatial Position Localization

With continuing reference to FIG. 13: in some embodiments, an EM transmitter is used, generating known EM fields of known frequencies and amplitudes, for example, low-frequency fields, for example 3-6 fields 1311A, 1311B, 1311C of different geometries at distinct frequencies below 500 Hz. The fields intersect to “tag” positions within a region, allowing each position to be distinguished according to the strengths of the various magnetic fields that pass through it, and/or their orientation.

In the example of FIG. 13, a single, optionally uniform coil 1301 is wrapped around a high permeability non-linear magnetic core 1301 at the tip of device 1300. This is then used as a sensor (e.g., connected to suitable sensing electronics via connections 1302, 1303) that measures the magnetic field at the sensor's tip. Since the transmitted fields 1311A-1311C are rather slow compared to the frequency at which sensor inductance can be measured (for example, the fields may vary with frequencies smaller than 500 Hz) the sensed fields can be measured with more than sufficient sampling rate to allow their decomposition into the different transmitted fields, for example using Discrete Fourier Transform (DFT) of the time-series or using correlation methods or using any other suitable method.

The sensed fields can then be used to solve the position and orientation of the sensor relative to the EM transmitter. With a single, spatially uniform coil 1305, the sensor 1310 may only sense the projection of the transmitter EM fields along its proximal-to-distal axis, which can provide a 5-DOF (5-Degrees of Freedom) solution of the sensor's tip in space relative to the transmitter. This amounts to 3-DOF position and 2-DOF orientation, where the roll angle of the sensor 1310 is missing.

### System for Multi-Field Spatial Localization and Shape Sensing

Reference is now made to FIG. 14, which schematically represents an endovascular tracking system 140, according to some embodiments of the present disclosure.

Sensor 141 is a multiplexed, thin and long (longitudinally extended) variable inductance sensor, corresponding, for example, to one or more of the embodiments of FIGS. 9-11, FIG. 13, or another sensor using principles of variable inductance (in response to magnetic fields, e.g., FIG. 13) and/or with varying inductance arranged along its length (e.g., FIGS. 9-11). Additionally, systems according with descriptions of FIG. 14 optionally include features described in relation to FIG. 5; for example in combinations also described in relation to FIG. 5.

In the example shown, sensor 141 is interconnected with external processing unit 500A and in particular readout controller 500B (e.g., via its connection terminals, for example, two connection terminals). Through the connection terminals, readout controller 500B drives sensor 141 with electrical currents used in magnetic field sensing. Moreover, readout controller 500B senses the magnetic field-reactive state of sensor 141, via the influence of the (variable) inductance of sensor 141 on properties of the electrical currents. The properties may include, for example, oscillation frequency of an oscillating circuit including the inductance of sensor 141, and/or amplitude of oscillation of such a circuit at one or more test frequencies. In particular, the properties are measured for a plurality of conditions distinguished by different driving current values.

Further operations attributed to the functioning of readout controller 500B optionally include processing of the current property measurements to indicate the state of a sensing region of sensor 141. The state is indicated as measurements, these measurements being indicative of local magnetic field (e.g., its strength) for a spatially distinguishable (although potentially in part overlapping) plurality of locations along the sensing region.

More particularly, influences on a current's properties indicate the total inductance (while subjected to that current) of sensor 141. The indications of total inductance are also referred to herein as “inductance information”, and this information is considered to be encoded in electrical signals which result from readout controller 500B driving current through sensor 141. Considered together with the driving currents used and other data about the construction of sensor 141, the inductance information encoded in these electrical signals can be transformed into measurements of local magnetic field for a plurality of locations along the sensing region. The locations are also distinguishable by their inductance density, or inductance they provide per unit of length. The difference in inductance density helps support the conversion of current property measurements to the measurements indicative of local magnetic field.

Still more particularly: the total inductance is in turn reactive to the driving currents, since in flowing through the inductive elements of sensor 141 those currents induce magnetization that temporarily affects the electromagnetic permeability of the longitudinally extended, high-permeability, and non-linear inductive core material of sensor 141. Since, furthermore, inductance density varies along the length of the sensing region, so does magnetization. This variation in permeability (or linear “inductance density”) is also referred to herein as inhomogeneous and/or non-uniform inductance density/permeability. In a first-order sense, this comprises baseline non-uniformity; that is, a non-uniformity independent of the non-linear changes in permeability next described.

The non-linearity of permeability change response as a function of magnetization means that different currents produce different relative amounts of inductance change in the plurality of locations along the sensing region.

After accounting for expected inductance changes resulting from self-generated magnetic fields, there may be residual deviations of the total inductance from, e.g., the zero-field inductance. The residual deviations from the reference are attributable to further influences on electromagnetic permeability, caused by magnetic fields generated externally to sensor 141 but also intersecting magnetic field sensor 141. Though initially measured as part of a total inductance, the residual deviations are indicative of integrated effects on individual parts of the inductance of sensor 141. Specifically, the inductance in each spatially distinguishable sensor location changes according to the magnetic field the sensor location interacts with.

Since, again, permeability changes are non-linear according to biases imposed by the particular current used to measure inductance, the individual changes are larger or smaller for the same local external magnetic field under conditions of different currents used in measuring inductance.

Informally, for a particular prove location, the particular set of magnetizing field strengths generated by a set of currents produces a “fingerprint”, defined, for example, as a function of change in permeability plotted against the measurement current. For a design of sensor 141 with a suitably inhomogeneous (non-uniform) baseline permeability, locations in the sensing region can be distinguished by this fingerprint. As one result of this: with different measurement currents applied, the distinguishable locations contribute respectively to changes from a baseline inductance in different relative amounts.

In some embodiments, the distinguishability of those sets of fingerprints is characteristic of what makes the locations distinguishable. For example, if two arbitrary regions are configured with inductance densities such that they share exactly the same set of fingerprints, they will sufficiently co-vary with changes in measurement current (even if exposed to different field strengths otherwise), as to become conflated in the measurement data as a single region.

Insofar as the construction of sensor 141 (i.e., with sufficiently distinct inductance densities) avoids creating this condition, the lumped inductance effects thus become potentially susceptible to mathematical separation,

Accordingly—using further inputs comprising, e.g., suitable calibration data and knowledge of how sensor 141 is constructed—mathematical processing performed, in some embodiments, by readout controller 500B, transforms the measurements of total inductance for different currents into measurements of local magnetic field for a plurality of locations along the sensing region of sensor 141. The processing may be carried out, for example, as described in relation to FIGS. 8-13 and in optionally in particular as described in relation to FIG. 9, or in an equivalent fashion.

Parenthetical to the hypothetical case of fully co-varying regions example given above: in some embodiments, degeneracy due to co-variance of inductance responses to magnetic fields for different regions (insofar as it is present, perhaps partially) is at least partially overcome. To begin with, even if the two regions statically experience different magnetic field strengths that remain mixed in a mathematical separation, the mixed value may vary with current in a way inconsistent with any single field strength. That, in turn, may allow deduction of separate magnetic field strengths which are consistent with the mixed value; though not, in the pure case, assignment of these to one or the other particular region.

Other information may allow this assignment, and/or be useful in determining the separate magnetic field strengths. For example, continuity constraints can be derived from knowing that the sensor comprises a continuous linear shape within continuously (over distance) varying fields. If, e.g., the above-mentioned “arbitrary regions” with the same inductance densities are also physically separated by other, distinguishable regions, then a solution may be estimated which jointly preserves a metric of continuity with neighboring regions, motivating a division of the jointly measured magnetic field strength into unequal contributions that “pay” for reducing discontinuity. In some embodiments, this approach is used to potentially create and/or increase mathematical separability of different locations along the sensing region. For example, applying the continuity constraint, a larger number of distinguishable regions may be separable within a given range of inductance densities among which those regions are distributed. In some embodiments, inductance densities of separable locations are arranged along the longitudinal extent of a sensor 141 such that differences with neighboring locations are maximized, and/or such that physical distances from the most similar locations are maximized. Together with this, constraints on continuity are applied. Error penalties for violating continuity constraints are reduced by re-balancing the relative assignment of field strengths for more distant pairs of locations with more similar inductance densities.

Optionally, at least portions of the system elements which perform functions of readout controller 500B are provided in an enclosure separate from external processing unit 500A. These portions of readout controller 500B then communicate with the processing unit 500A, at least to convey data in some form from sensor 141. Insofar as further processing is performed after this conveyance to perform the transformation into local magnetic field measurements, processing unit 500A also acts as a portion of readout controller 500B.

Optionally, portions of readout controller 500B responsible receive commands from processing unit 500A governing control of readout to suitably drive and directly measure from sensor 141. Additionally or alternatively, the portions of readout controller 500B responsible for driving and direct measurement are separately (e.g., manually) configurable.

External processing unit 500A may incorporate further processing features; for example as described in relation to FIG. 5, herein.

Also provided, in some embodiments, are EM field control unit 503, interconnected with a plurality of field generators 502 (e.g., magnetic field generators). The field generators 502 generate distinguishable magnetic fields 151A-151C; for example, distinguishable according to a frequency of their generation. Field generators 502 are arranged around navigational region of interest 505, comprising, for example, a portion of a human body (e.g., a cranium). The local strengths and/or directions of fields 151A-151C are at least partially uncorrelated with each other, so that field measurements made within a region of suitable size to overcome signal-to-noise limitations are distinguishing of that region from adjoining regions, preferably in any direction.

In some embodiments, sensor 141 is part of (e.g., attached to or integrated within) another device, for example, endoluminal device 141A, which comprises a long, thin body sized, shaped, flexible, pushable, and otherwise configured for endoluminal navigation that advances a tip of endoluminal device 141A through narrow lumens of the body. Endoluminal device 141A optionally has a length to diameter ratio greater than 100, and typically a much larger such ratio; e.g., in the range of several hundred to about a thousand (e.g., 1 meter long, 1 mm in diameter). Sensor 141 may be a body-integrated sensor of endoluminal device 141A, in the sense described for the single-coil embodiment of FIG. 13.

Sensor 141, as part of a navigable endoluminal device 141A, is shown introduced into navigational region of interest 505, via a vasculature 506, of which a restricted portion is shown for purposes of illustration. Endoluminal device 141A may be, for example, a guidewire, catheter, or other long and thing (longitudinally extended) tool suitable for insertion into longitudinally navigated anatomical channels such as blood vessels and/or airways.

Among the potential advantages of sensor 141 is its optionally very small cross-section. Its inductive core (e.g., a wire of having variable permeability in response to magnetic fields) may be roughly equal to or less than the diameter which is provided anyway to non-sensing portions of endoluminal device 141A. It is wrapped with fine coil windings, preferably significantly thinner still (e.g., 40-54 AWG) which potentially increase this diameter by only a small fraction (e.g., less than about 40%, less than about 33%, less than about 25%, or less than about 15%). For example, 44 AWG wire is about 50 μm in diameter, so that it adds a total thickness of about 100 μm. The overall diameter may be, for example 1 mm or less, 750 μm or less, 500 μm or less, 400 μm or less, 350 μm or less, or 250 μm or less. The diameter may be different at different positions along the proximal-distal axis of the sensor 141, e.g., between diameters in a range between about 1.5 mm and 250 μm. For example, the inductive core may be tapered from about 900 μm on a proximal side and about 300 μm on a distal side, with the added coil windings adding approximately another 100 μm for a total width range between about 1000 μm and about 400 μm. The ratio between largest and smallest diameters along the sensing region of sensor 141 may be, for example, at least 1.5, 2, 2.5, or 3. The sensor may itself provide the mechanical properties which provide the mechanical navigability properties device 141A along its length, so that other elements alongside it are unnecessary. The variable permeability material may be provided alone as the core material along the extent of sensor 141, or it may be mixed in with other material, e.g., as part of one or more alloys, within a matrix (e.g., a polymer matrix), as strands of different compositions, or in another manner.

Another potential benefit of such sensors is design simplicity and cost, with corresponding potential advantages for use as a disposable device. For example, in certain applications a plurality of magneto-inductive fully tracked “guidewires” can be introduced to a patient's organ and provide a fully tracked real-time skeleton of that organ for certain uses, such as modeling the deformation of that organ during a medical procedure.

Within the context of a minimally invasive medical procedure relying on endoluminal (e.g., endovascular, or more particularly, neurovascular or cardiovascular) guidewire navigation, there may be more than one sensing modality in place. For example, external imaging device 510 is optionally provided, which is optionally an X-ray imager. Ionizing radiation-based imaging, while well-known and commonly used as a way of monitoring endovascular instrument positioning, has associated exposure risks. In some embodiments of the present disclosure, position determination using sensor 141 allows reduction of ionizing radiation exposure, e.g., reduced imaging frame rates (e.g., 5 Hz instead of 30 Hz, or another factor of frame rate reduction). In some embodiments, use of fluoroscopy is reduced to obtaining occasional recalibration and/or verification images, for example every few seconds or even minutes, with navigation proceeding in between guided by probe shape and position measurements. To maintain an acceptable level real-time feedback, interpolation of probe position through the “missing” frames optionally uses spectrally multiplexed position sensing to infer changes in position, e.g., from a previous X-ray “key frame” image. Alternative and/or additional processing operations using the mixed data stream may be used to provide feedback (e.g. displays shown using user interface 520) which help an operator of device 141A maintain an uninterrupted awareness of the results of their actions to navigate (e.g., steer, rotate, push, and/or pull) or otherwise operate device 141A.

With respect to optional robotic driver 521 provided in some embodiments corresponding to FIG. 14, features described in relation to robotic driver 521 of FIG. 5 also apply. In overview: in some embodiments, robotic driver 521 actuates movements of endoluminal device 141A for navigation as such. In particular, selection of these movements may be based on currently observed shape and/or position of sensor 141, and more particularly, based on the relationship of the current shape/position of sensor 141 to the relative location of targets such as vascular bifurcation branches. Additionally or alternatively, in some embodiments, movements may be actuated to help sense the present state and/or responsiveness to input of endoluminal device 141A. In particular, known small actuation inputs are optionally compared to observed corresponding changes in the shape and/or position of sensor 141. Optionally, larger movements are selected, suggested, and/or performed, based on these observed correspondences and the relative positioning of one or more navigation targets.

### Variations of the Shape/Position Sensor

With continuing reference to FIG. 14, sensor 141 provides, in some embodiments, a flexible curve sensor comprising a single continuous coil wrapped around a flexible magnetic core, and usable to measure a plurality of magnetic fields at plurality of positions along its length, for example 4 or 8 or more fields and 4 or 8 or more positions. By sensing a plurality of magnetic fields, a system 140 incorporating sensor 141 may then solve (e.g., using processing unit 500A) for each of the plurality of positions a position and orientation of the sensor's curve relative to one or more EM transmitters 502. This can provide a full shape localization of the flexible sensor 141 relative to the EM transmitters 500A.

More particularly, in some embodiments, EM shape sensor 141 comprises a long continuous coil wrapped around a high permeability non-linear flexible magnetic core, for example, such as a supermalloy wire. The coil may have varying winding pitch and can comprise, for example, a 40-44 AWG copper or gold wire. The wire can be wrapped from left to right and then in a second layer from right to left such that the 2 terminals of the coil are available on the same proximal side of the sensor and are available for connection to the sampling electronics.

In some embodiments of the present disclosure, only the tip of sensor 141 is wrapped with a short coil, for example, a 3 mm-10 mm coil, to sense DC and/or time-oscillating magnetic field strength at the tip of the sensor and to optionally provide localization of the sensor's tip relative to an EM transmitter.

In other embodiments, only the distal end of sensor 141 is wrapped with a medium length coil, for example, a 10 mm-40 mm varying-pitch coil, (e.g. varying in pitch as depicted in FIG. 9), to sense a plurality of DC and/or time-oscillating magnetic fields at the distal end of the sensor 141 and to optionally provide data used by system 140 for curve localization (shape sensing) of the sensor 141 distal end relative to one or more EM transmitters 502. Sensor 141 may provide readings distinguishing, e.g., 2-4 positions along its coil-wrapped distal length.

In another embodiment, the distal part of sensor 141 is wrapped with a continuous coil which can optionally be of varying pitch, for example, a 40 mm-200 mm varying-pitch coil, for example, as depicted in FIG. 11, to sense a plurality of DC and/or time-oscillating magnetic fields at the distal part of the sensor and to optionally provide full curve localization (shape sensing) of the sensor's distal part relative to an EM transmitter. The sensor may provide readings distinguishing magnetic fields at e.g., 2-19 positions along its coil-wrapped distal part, which can be used to compute a plurality of positions and orientations along the sensor's curve to provide the sensor's curve position and orientation.

### Variants of a Shape Sensing System

With further reference to FIG. 14, it should be understood that sensor 141 is optionally provided separately from an endoluminal device 141A, and may optionally be configured for operation as a separate device in its own right, or together with another device which is not an endoluminal device 141A (at least, not in the sense of being a medical endoluminal device). With reference to FIG. 14 as a baseline, the elements of a shape-sensing navigation system more generally may be understood to exclude medical endoluminal device 141A. Furthermore, external imaging device 510 is also optionally excluded, including in a medical endoluminal device implementation of system 140.

However, imaging device 510 may nevertheless be provided in some applications. Optionally it is provided other than as shown (i.e., within rather than outside the space that sensor 141 traverses). For example, imaging device 510 may be provided an optical imaging device which is physically interconnected with and moved along by navigation of sensor 141 itself.

For example, sensor 141 may be used to assist in manufacturing, inspection, and/or repair operations which involve the traversal of deep but confined spaces, e.g., using small access ports. In particularly, there is potential suitability of shape data for use in robotically guided navigation of such spaces, since the data may be directly produced in spatial coordinates. This potentially eliminates a need to directly overcome the numerous feature recognition issue which may arise in image-guided navigation approaches. In some embodiments, a largely image-guided navigation approach may be supplemented using shape sensing, e.g., to reduce computational complexity of traversing crowded and/or visually complicated environments or portions thereof.

Interconnection of processing unit 500A and user interface 520 is optional, at least in the general case. For example, a robotic navigation system may forgo a user interface which provides direct indications of shape sensing in normal use.

The EM field control unit 503 and field generators 502 are not necessarily provided together with a system 140, e.g., they may be separately provided as part of another magnetic sensing system, or sensing may be occurring in an environment which is magnetically “rich” for another reason, e.g., in the context of electrical power generation and/or transmission applications. In some applications, one or more permanent magnets may be sufficient to establish magnetic fields which determine at least device orientation, and potentially also shape in whole or in part, depending e.g., on how many magnets are used, and other constraints such as limitation of the sensor 141 to move in a restricted near-planar volume, near-cylindrical volume, or other restrictive and well-characterized volume shape.

These types of alterations should also be understood to apply, changed if and as necessary, to embodiments of systems using sensors such as are described in FIG. 5. In particular, references to magnetoresistive type sensors such as sensor 501 of FIG. 5 may substituted for references in this section to magneto-inductive type sensors such as sensor 141 of FIG. 14, at least insofar as system configuration variations disclosed do not rely on or make particular reference to sensor characteristics which differ between the two sensor types.

### Spatial Localization Enhancements

In some embodiments, an EM transmitter (comprising control unit 503 and field generator 502 of FIG. 14) used generates a small number of different EM fields (for example, 3-6 fields). FIG. 14 shows three fields 151A-151C; this number should be understood as a non-limiting example.

The field number can alternatively be large (for example, 20-30 fields). As the number of generated EM fields increases, the number of sensed fields increases accordingly and the conversions between the sensed fields to a position and orientation solution of the sensor may become more accurate and robust. However, it is sometimes necessary to keep the number of transmitted fields in the lower of these two regimes; for example, to reduce power consumption in the EM transmitter, reduce setup complexity, or another reason. With just 6 transmitted EM fields, each discrete magnetic field time-series along the sensor's curve can be converted individually to a position and orientation of that point relative to the transmitter. The system may remain at least somewhat exposed to noise and/or bias errors, however.

In some embodiments, shape, smoothness, and/or distance constraints derived from the construction of a sensor 141 (e.g., as a fixed-length, linear device) are used to potentially improve accuracy and/or robustness of the solution of the sensor's shape from measurements made using it.

For example, in some embodiments of the present disclosure: instead of solving the position and orientation of each discrete point along the sensor's curve individually, the complete shape of the sensor may be solved (e.g., by processing unit 500A) as a whole, under shape and smoothness constraints. The problem of solving a position and orientation (for example, 5-DOF) of points along the sensor's length can be viewed as an optimization problem. The goal of the optimization is to minimize the error between the sensed magnetic fields as a time series (or as signed amplitudes after a DFT conversion) and the known generated magnetic fields (the model). By finding the position and orientation of each discrete point along the sensor's length under the known generated magnetic fields which explain the measurements, the position and orientation of each discrete point is solved.

By imposing shape and smoothness constraints of the solved positions and orientations of the sensor's curve, the dimensionality of the optimization problem is reduced and the number of measurements can be reduced accordingly. For example, in FIG. 10 an EM shape sensor is depicted which comprises 8 discrete sub-coils connected in series. Using the methods described above, the sensor can convert the sensed inductive curve into 8 separate magnetic field measurements, corresponding to the 8 discrete coils. Instead of then solving 8 individual positions and orientations, the complete sensor curve may be solved under the known transmitted EM fields and by imposing shape and smoothness constraints, such that the magnetic field measurements are explained. For example, the pre-calibrated length between the discrete coils may be used to force the solver to solve 8 discrete positions along the curve which conform to the known lengths between the discrete coils. Additionally, the solver implemented by processing unit 500A may “punish” un-smooth position and orientation solutions between neighboring coils. This may allow the sensor to be operated under a small number of transmitted EM fields, for example, using just 3 fields, which may be insufficient when positions and orientations are solved individually, but can be sufficient once the sensor's shape is solved as a whole.

In some embodiments of the present disclosure, the solution approach implemented by processing unit 500A is further generalized in the following manner: instead of decomposing the sensed inductance curve by the sensor to individual magnetic field measurements (for example, 8 different measurements), the sensor may solve the full sensor curve in space relative to the transmitter, under shape and smoothness constraints, such that the predicted inductance curve (according to a pre-calibrated model) of the predicted sensor's full curve position and orientation in space would yield the measured inductance curve. This may further reduce the dimensionality of the optimization problem and correct for potential errors in the conversion between the measured inductance curve and individually sensed magnetic fields. By imposing constraints (such as shape and smoothness constraints) and under the assumption that the sensor resides within some curved location at the proximity of a known and calibrated EM transmitter, only certain inductance curves are possible (over time) which are indicative under a constrained curved shape of the EM shape sensor in space.

### Spatial Localization Enhancements to Calibration

In some embodiments of the present disclosure, the exact inductance to magnetic field relationship of a sensor 141 (FIG. 14) may not be known in advance, and may need to be calibrated. Furthermore, the exact relationship between some curved position and orientation of the flexible sensor in space relative to an EM transmitter and the sensed inductance curve (over time) may be unknown. Furthermore, the exact frequencies and amplitudes of the transmitted EM fields may be unknown in advance. To solve this, a calibration process may be used where the flexible sensor is positioned in a plurality of curved positions and orientation in space relative to the transmitter.

The raw sensor data may be collected over time; for example, the full inductance curves over time may be collected. A model is then used which can include a plurality of variables, such as for example: the frequencies and amplitudes of the transmitted EM fields, the curved positions and orientations of the flexible EM shape sensor 141 over time, the relationship between externally applied magnetic fields along the sensor's curve and the sensed inductance curve by the sensor etc. These variables can then be solved, for example, under imposed shape and smoothness constraints of the curved EM sensor. As long as the dimensionality of the measurements (that is, the inductance curves sensed by the sensor over time) exceeds the dimensionality of the variables, then the variables can be solved in a calibration process using an optimization process. This can provide a very accurate unsupervised calibration of the EM shape sensor. In a more supervised setting, the dimensionality of the variables can be reduced, for example, by placing the curved EM sensor in known curved positions and orientations relative to the EM transmitter, thus removing the curved positions and orientations from the list of unknown variables during calibration to make the optimization process more robust. The result of the calibration process is a prediction model which can provide predictions for the sensed inductance curves of the sensor for a given curved position and orientation of the EM shape sensor 141 relative to the transmitter(s) 502. This prediction model can then be used to solve the curved position and orientation of the sensor 141 in real-time, providing shape sensing of the sensor 141, e.g., as described above.

### GENERAL

It is expected that during the life of a patent maturing from this application many relevant variably resistive, variably inductive, and/or high-permeability materials will be developed; the scope of the term variably resistive material, variably inductive material, and/or high-permeability material is intended to include all such new technologies a priori.

As used herein with reference to quantity or value, the term “about” means “within ±10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the present disclosure may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments may be presented with reference to a range format. It should be understood that the description in range format is merely for convenience and brevity and should not be construed as an inflexible limitation on the scope of descriptions of the present disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although descriptions of the present disclosure are provided in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

It is appreciated that certain features which are, for clarity, described in the present disclosure in the context of separate embodiments, may also be provided in combination in a single embodiment. Conversely, various features, which are, for brevity, described in the context of a single embodiment, may also be provided separately or in any suitable subcombination or as suitable in any other described embodiment of the present disclosure. Certain features described in the context of various embodiments are not to be considered essential features of those embodiments, unless the embodiment is inoperative without those elements.

It is the intent of the applicant(s) that all publications, patents and patent applications referred to in this specification are to be incorporated in their entirety by reference into the specification, to the same extent as if each individual publication, patent or patent application was specifically and individually noted when referenced that it is to be incorporated herein by reference. In addition, citation or identification of any reference in this application shall not be construed as an admission that such reference is available as prior art to the present disclosure. To the extent that section headings are used, they should not be construed as necessarily limiting. In addition, any priority document(s) of this application is/are hereby incorporated herein by reference in its/their entirety.

1. A system configured to sense a magnetic field at one or more locations along a sensing region of a flexible and elongated probe body, the system comprising:
the elongated probe body;
a flexible sensing circuit comprising inductance density along the sensing region;
a processor configured to:
sense total inductances of the flexible sensing circuit for each of a plurality of different electrical current values passed through the circuit; and
determine sensed magnetic field for the one or more locations along the sensing region.

2. The system of claim 1, wherein the one or more locations along the sensing region comprise locations with respectively different inductance densities, such that the different electrical current values generate different respective internal magnetization field strengths in each said location.

3. The system of claim 2, wherein the respectively different internal magnetization field strengths result in different internal inductance density variations in each of the one or more locations along the sensing region.

4. The system of claim 3, wherein the different internal inductance density variations adjust the sensing inductance density variations to different functions of sensed magnetic field strength in each of the one or more locations.

5. The system of claim 4, wherein the processor is configured to:
receive a model of the non-uniform distribution of baseline inductance density; and
calculate strengths of the sensed magnetic field to be consistent with both the internal magnetization field strengths, and the different functions of sensed magnetic field strength in each of the one or more locations along the sensing region.

6. The system of claim 1, wherein the flexible sensing circuit comprises inductor coil windings that wrap a magnetic inductor core which changes non-linearly in electromagnetic permeability as a function of total local magnetic field strength.

7-9. (canceled)

10. The system of claim 1, wherein non-uniformity of a baseline inductance density is provided using a non-uniform density of coil windings along the sensing region.

11. The system of claim 10, wherein the non-uniform density of coil windings along the sensing region comprises gradually changing coil spacing along the sensing region.

12. The system of claim 10, wherein the non-uniform density of coil windings along the sensing region comprises discrete sections of coil along the sensing region, each section having a different number of coil windings.

13. The system of claim 12, wherein different numbers of coils windings are provided as coils in different numbers of layers.

14-20. (canceled)

21. The system of claim 10, wherein non-uniformity of the baseline inductance density is provided using a range of different diameters between the largest and smallest diameter as diameters of different inductive coil windings contributing to the total inductance.

22. The system of claim 10, wherein non-uniformity of the baseline inductance density is provided using variations in composition of a magnetic core of the inductor coils contributing to the total inductance.

23. The system of claim 1, wherein the processor is configured to calculate strengths of sensed magnetic field for the one or more locations along the sensing region by solving a system of non-linear equations, each respectively defined using the measurements of the variable electrical inductance and their corresponding electrical current values.

24. The system of claim 1, comprising a magnetic field generating controller and a plurality of magnetic field generating transducers generating a respective plurality of magnetic fields.

25. The system of claim 24, wherein the plurality of magnetic fields are generated at different frequencies, and distinguished by a readout controller that provides the measurements using differences in their frequencies.

26. The system of claim 24, comprising a processing unit configured to determine a shape and position of the sensing circuit, using the measurements of sensed magnetic field strengths.

27. (canceled)

28. The system of claim 1, wherein components of the sensing circuit also provide mechanical properties of the elongated probe body at a distal end thereof, the mechanical properties being suitable for advancing the sensing circuit by insertion of the elongated probe body into a body cavity a distance of at least 50 mm, to reach a body cavity region having an inner diameter of 5 mm or less.

29. The system of claim 1, wherein the provided measurements are constrained by criteria requiring one or more of the group consisting of: smoothness of the sensed magnetic field along the sensing region, preservation of distances between sensing regions, and/or preservation of known positions of sensing regions.

30. The system of claim 6, wherein:
the inductor coil windings surround an inductor core material; and
the processor is configured to:
access at least one impedance curve defining changes in inductance density as a function of magnetization of the inductor core material, and
calculate the strengths of sensed magnetic field for the one or more locations along the sensing region, using an impedance curve.

31. A method of sensing a magnetic field at a plurality of locations along a flexible elongated probe body, the method comprising:
sensing total inductances of a flexible sensing circuit for each of a plurality of different electrical current values passed through the circuit, wherein the circuit comprises varying inductance density; and
determining the sensed magnetic field for a plurality of mutually distinguished inductance density locations along the flexible sensing circuit.

32. The method of claim 31, comprising using the sensed magnetic field to determine one or more positions along the flexible elongated probe body.

33. The method of claim 31, comprising using the sensed magnetic field to determine a shape of the flexible elongated probe body.

34-43. (canceled)

44. The system according to claim 1, wherein the flexible sensing circuit comprises variable inductance density.

45. The system according to claim 1, wherein the locations are a plurality of mutually distinguished inductance density locations along the flexible sensing circuit.
